(19) United States
(12) Patent Application Publication
(10) Pub. No.: US 2019/0001311 A1
(43) Pub. Date: Jan. 3, 2019

(54) ZEOLITIC 3D SCAFFOLDS WITH TAILORED SURFACE TOPOGRAPHY FOR METHANOL CONVERSION WITH LIGHT OLEFINS SELECTIVITY

(71) Applicants: Ali A. Rownaghi, Rolla, MO (US); Xin Li, Rolla, MO (US); Fateme Rezaei, Rolla, MO (US)

(72) Inventors: Ali A. Rownaghi, Rolla, MO (US); Xin Li, Rolla, MO (US); Fateme Rezaei, Rolla, MO (US)

(73) Assignee: The Curators of the University of Missouri, Columbia, MO (US)

(21) Appl. No.: 16/023,997

(22) Filed: Jun. 29, 2018

Related U.S. Application Data
(60) Provisional application No. 62/527,251, filed on Jun. 30, 2017.

(51) Int. Cl. B01J 29/00; B01J 21/16; B01J 29/85; B01J 29/40; B01J 29/48; B01J 29/46; B01J 35/04; B01J 35/10; C07C 4/06

(52) U.S. Cl. CPC B01J 29/005; B01J 21/16; B01J 29/85; B01J 29/405; B01J 29/48; B01J 29/46; C07C 2529/85; B01J 35/1038; B01J 35/1042; C07C 4/06; C07C 2529/48; C07C 2529/46; C07C 2529/40; B01J 35/04

(57) ABSTRACT

The present disclosure relates to 3D printed zeolite scaffolds. The zeolite scaffolds can be used as a catalyst for methanol to olefin (MTO) conversion and hydrocarbon cracking processes.

### CROSS REFERENCE TO RELATED APPLICATIONS

This application claims the benefit of U.S. Provisional Application No. 62/527,251, filed Jun. 30, 2017, the disclosure of which is hereby incorporated by reference in its entirety.

### FIELD OF THE INVENTION

The present application is directed to catalysts for methanol to olefin (MTO) conversion. Specifically, the present disclosure is directed to MTO conversion with light olefin selectivity using zeolite 3D scaffolds having tailored surface topography.

### BACKGROUND OF THE INVENTION

Methanol to olefin (MTO) conversion is a significant process in the industrially demanding production of light olefins via natural gas, coal, or even biomass. Zeolites are an important catalyst used in MTO conversion due to its tunable acidity, unique porosity and special configuration. In particular, SAPO-34 silico-aluminophosphate (CHA structure) is recognized as an efficient catalyst to produce a high selectivity towards light olefins (ethylene and propylene) due to its proper acid strength and small pore channels (3.73 Å×3.73 Å). However, a high rate of deactivation is usually observed for SAPO-34 due to coke formation in the confined cavities of the catalyst. Therefore, as a compromise, ZSM-5 zeolite (MFI structure) with larger channels (4.70 Å×4.46 Å) is often used, despite its characteristic lower light olefin selectivity, in order to extend the lifetime of the catalyst.

Monolithic catalysts, generally regarded as a block of structured material containing various types of interconnected or separated channels, were introduced in the 1970s. Monoliths are generally characterized as having high thermal stability, good mechanical integrity, good mass transfer characteristics, a low pressure drop compared with packed-bed reactors, and suitable performance in many processes. Monolithic catalysts are advantageous in multiphase reactions such as the cleaning of automotive exhaust gases and industrial selective catalytic reduction (SCR). In at least some instances, monolithic catalysts may offer an alternative to the slurry reactor. Monoliths have also attracted researchers as a medium for the utilization of absorbents made of activated carbon, zeolites, and metal-organic frameworks (MOFs).

Generally, monoliths are fabricated using an extrusion process. Unique dies are developed with specific sizes and shapes, through which a mixture of raw materials, such as powders, binders, and plasticizers are extruded. The desired monoliths may be obtained after drying and firing. MTO conversion catalysts having selectivity for light olefins and resistance to fouling or extended catalyst lifetimes are desirable.

### SUMMARY OF THE INVENTION

One aspect of the present disclosure is directed to a zeolite coated monolith article. The zeolite coated monolith article comprises an uncoated monolithic support structure including walls having a honeycomb structure comprising ammonia-ZSM-5 powder (SiO₂/Al₂O₃) and bentonite clay; and a porous coating disposed directly upon the uncoated monolithic support structure.

Another aspect of the present disclosure is directed to a method of producing a zeolite monolith article. The method comprises (a) calcinating the ammonia-ZSM-5 powder (SiO₂/Al₂O₃) at temperature from about 500° C. to about 1100° C. for about 2 hours to about 8 hours to produce a mixture comprising ZSM-5 zeolite and Y zeolite; (b) mixing the mixture comprising mixture of ZSM-5 zeolite and Y zeolite with bentonite clay and water to produce a homogenous slurry; and (c) 3D-printing the homogenous slurry on an alumina substrate to produce the zeolite monolith.

An additional aspect of the present disclosure is directed to a process for converting methanol to one or more light olefins (MTO). The process comprises contacting methanol, under deoxygenation conditions, with a catalyst comprising a zeolite monolith, wherein the zeolite monolith comprises an uncoated monolithic support structure including walls having a honeycomb structure comprising ammonia-ZSM-5 powder (SiO₂/Al₂O₃) and bentonite clay, and a porous coating disposed directly upon the uncoated monolithic support structure.

In yet another aspect of the present disclosure is directed to a method for cracking a hydrocarbon. The process comprises contacting the hydrocarbon, under cracking conditions, with a catalyst comprising a zeolite monolith, wherein the zeolite monolith comprises an uncoated monolithic support structure including walls having a honeycomb structure comprising ammonia-ZSM-5 powder (SiO₂/Al₂O₃) and bentonite clay; and a porous coating disposed directly upon the uncoated monolithic support structure.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the optical image of the 3D-printed ZSM-5 monoliths with various dopants.

FIG. 2 depicts XRD patterns of the 3D-printed ZSM-5 monolith and its metal-doped counterpart.

FIG. 3 depicts SEM images for ZSM-5 and M/ZSM-5 monolith samples.

FIG. 4 depicts the schematic of the pores derived from the decomposition and removal of methyl cellulose.

FIG. 5A and FIG. 5B depict FTIR spectra of the 3D-printed ZSM-5 monolith and its metal-doped counterparts in the range of (FIG. 5A) 2000-400 cm⁻¹ and (FIG. 5B) 4000-3000 cm⁻¹.

FIG. 6A and FIG. 6B depict representation of two ways of metal doped in zeolite: (FIG. 6A) introduced to Al—OH—Si hydroxyl group (internal strong Brønsted acid); (FIG. 6B) introduced to Al—OH hydroxyl group (external weak Brønsted acid).

FIG. 7A, FIG. 7B, FIG. 7C, FIG. 7D, FIG. 7E, FIG. 7F, FIG. 7G, FIG. 7H, and FIG. 7I depict N₂ physisorption isotherms of bare ZSM-5 (FIG. 7A) and Ce/ZSM-5 (FIG. 7B), Cr/ZSM-5 (FIG. 7C), Cu/ZSM-5 (FIG. 7D), Ga/ZSM-5 (FIG. 7E), La/ZSM-5 (FIG. 7F), Mg/ZSM-5 (FIG. 7G), Y/ZSM-5 (FIG. 7H), and Zn/ZSM-5 (FIG. 7I) samples with BJH adsorption pore size distribution in inset.

FIG. 8 depicts NH₃-TPD profiles of the as-prepared samples.

FIG. 9 depicts H₂-TPR profiles of the as-prepared samples.

FIG. 10A, FIG. 10B, FIG. 10C, FIG. 10D, FIG. 10E, FIG. 10F, FIG. and 10G depict EDS analysis and corresponding mapping of the elements in the 3D-printed monolith samples (FIG. 10A) ZM, (FIG. 10B) CrZM, (FIG. 10C) GaZM, (FIG. 10D) LaZM, (FIG. 10E) MgZM, (FIG. 10F) YZM, and (FIG. 10G) ZnZM.

FIG. 11A and FIG. 11B depict compressive strength of the as-prepared 3D-printed monolith samples: (FIG. 11A) Compressive stress versus strain, (FIG. 11B) the critical compressive stress of various composition.

FIG. 12A, FIG. 12B, FIG. 12C, FIG. 12D, FIG. 12E, FIG. 12F, FIG. 12G, FIG. 12H, and FIG. 12I depict the conversion of methanol (X_{MeOH}) as a function of time on stream over all the investigated catalysts ((FIG. 12A) ZM, (FIG. 12B) Ce, (FIG. 12C) Cr, (FIG. 12D) Cu, (FIG. 12E) Ga, (FIG. 12F) La, (FIG. 12G) Mg, (FIG. 12H) Y, and (FIG. 12I) Zn) at 623 K, WHSV: 0.35 h⁻¹.

FIG. 13A, FIG. 13B, FIG. 13C, FIG. 13D, FIG. 13E, FIG. 13F, FIG. 13G, FIG. 13H, and FIG. 13I depict the selectivity to hydrocarbons as a function of time on stream over all the investigated catalysts ((FIG. 13A) ZM, (FIG. 13B) Ce, (FIG. 13C) Cr, (FIG. 13D) Cu, (FIG. 13E) Ga, (FIG. 13F) La, (FIG. 13G) Mg, (FIG. 13H) Y, and (FIG. 13I) Zn at 623 K, WHSV: 0.35 h⁻¹; Reaction temperature: 623 K, WHSV: 0.35 h⁻¹. The legend at the bottom of the figure is related to all the samples and color-independent.

FIG. 14 depicts TGA (lower) and DTA (upper) profiles of the spent catalysts after 24 h of methanol conversion at 673K.

FIG. 15 depicts Zeolite monoliths (M1) extruded by the 3D printer and microscopic image of the channels.

FIG. 16 depicts the schematic of the fixed-bed reactor setup.

FIG. 17 depicts XRD patterns of the bare (M1, M2) and SAPO-coated (SPM1, SPM2) 3D-printed monoliths.

FIG. 18A, FIG. 18B, FIG. 18C, FIG. 18D, FIG. 18E, and FIG. 18F depict SEM image of (FIG. 18A) the surface of M1; (FIG. 18B) the cross section of SPM1; (FIG. 18C) the surface of M2; (FIG. 18D) cross sectional view of SPM2; (FIG. 18E) top view of SPM1; (FIG. 18F) parent HZSM-5 powder.

FIG. 19A, FIG. 19B, FIG. 19C, FIG. 19D, FIG. 19E, and FIG. 19F depict N₂ physisorption isotherms of (FIG. 19A) pure HZSM-5 and silica, (FIG. 19C) M1 and SPM1, (FIG. 19E) M2 and SPM2; PSD of corresponding monoliths: (FIG. 19B) pure HZSM-5 and silica, (FIG. 19D) M1 and SPM1, (FIG. 19F) M2 and SPM2. The PSD was derived from BJH method using the adsorption branch of the N₂ isotherm.

FIG. 20 depicts schematic of monolith surface topology and porosity affected by SAPO-34 crystal growth.

FIG. 21A and FIG. 21B depict compressive strength of 3D-printed monoliths: (FIG. 21A) Compressive load versus specimen displacement, (FIG. 21B) the critical compressive load of M1, M2, and M3 monoliths.

FIG. 22A and FIG. 22B depict NH₃-TPD profiles (FIG. 22A) and py-FTIR spectra (FIG. 22B) of the HZSM-5 powder, the 3D-printed monolith and the counterparts with SAPO-34 crystals.

FIG. 23A, FIG. 23B, FIG. 23C, and FIG. 23D depict the conversion of methanol (X_{MeOH}) as a function of time on stream over all the investigated catalysts at various reaction conditions (FIG. 23A) Reaction temperature: 623 K, WHSV: 0.35 h⁻¹; (FIG. 23B) Reaction temperature: 623 K, WHSV: 1.06 h⁻¹; (FIG. 23C) Reaction temperature: 673 K, WHSV: 0.35 h⁻¹; (FIG. 23D) Reaction temperature: 673 K, WHSV: 1.06 h⁻¹.

FIG. 24A, FIG. 24B, FIG. 24C, and FIG. 24D depict the selectivity to ethylene as a function of time on stream over all the investigated catalysts at various reaction conditions (FIG. 24A) Reaction temperature: 623 K, WHSV: 0.35 h⁻¹; (FIG. 24B) Reaction temperature: 623 K, WHSV: 1.06 h⁻¹; (FIG. 24C) Reaction temperature: 673 K, WHSV: 0.35 h⁻¹; (FIG. 24D) Reaction temperature: 673 K, WHSV: 1.06 h⁻¹.

FIG. 25A, FIG. 25B, FIG. 25C, and FIG. 25D depict the selectivity to propylene as a function of time on stream over all the investigated catalysts at various reaction conditions (FIG. 25A) Reaction temperature: 623 K, WHSV: 0.35 h⁻¹; (FIG. 25B) Reaction temperature: 623 K, WHSV: 1.06 h⁻¹; (FIG. 25C) Reaction temperature: 673 K, WHSV: 0.35 h⁻¹; (FIG. 25D) Reaction temperature: 673 K, WHSV: 1.06 h⁻¹.

FIG. 26 depicts TGA (lower) and DTA (upper) profiles of the spent catalysts after methanol dehydration, Reaction temperature: 673 K, WHSV 0.35 h⁻¹.

FIG. 27A and FIG. 27B depict (FIG. 27A) a schematic of the MTO reaction pathway [60] and (FIG. 27B) the occurrences over the zeolite monolith and powder in this work.

FIG. 28 depicts ²⁹Si MAS NMR spectra of the fresh and spent catalysts after 15 hours of reaction (673 K, WHSV: 1.06 h⁻¹).

### DETAILED DESCRIPTION OF THE INVENTION

Applicants have discovered that 3D-printed zeolite monoliths have low fouling rates and extended catalyst lifetime when used as a catalyst in methanol to olefin (MTO) conversion reactions.

Additional aspects of the invention are described below.

### (I) Zeolite Monolith

One aspect of the present disclosure encompasses a zeolite coated monolith article, comprising an uncoated monolithic support structure including walls having a honeycomb structure comprising ammonia-ZSM-5 powder (SiO₂/Al₂O₃) and bentonite clay; and a porous coating disposed directly upon the uncoated monolithic support structure.

Other aspects of the invention are described in further detail below.

### (a) Composition

In general, the uncoated monolithic support structure comprises zeolite ammonia-ZSM-5 powder (SiO₂/Al₂O₃) and bentonite clay.

(i) Zeolite Ammonia-ZSM-5 Powder (SiO₂/Al₂O₃)

In an embodiment, the uncoated monolithic support structure may comprise from about 30 wt. % to about 95 wt. % zeolite ammonia-ZSM-5 powder (SiO₂/Al₂O₃). In some embodiments, the uncoated monolithic support structure may comprise from about 30 wt. % to about 95 wt. %, about 40 wt. % to about 90 wt. %, or from about 45 wt. % to about 90 wt. % zeolite ammonia-ZSM-5 powder (SiO₂/Al₂O₃).

In additional embodiments, the uncoated monolithic support structure may comprise from about 40 wt. % to about 50 wt. % zeolite ammonia-ZSM-5 powder (SiO₂/Al₂O₃). In other embodiments, the uncoated monolithic support structure may comprise about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, or about 50 wt. % zeolite ammonia-ZSM-5 powder (SiO₂/Al₂O₃).

In additional embodiments, the uncoated monolithic support structure may comprise from about 80 wt. % to about 90 wt. % zeolite ammonia-ZSM-5 powder (SiO₂/Al₂O₃). In other embodiments, the uncoated monolithic support structure may comprise about 80 wt. %, about 81 wt. %, about 82 wt. %, about 83 wt. %, about 84 wt. %, about 85 wt. %, about 86 wt. %, about 87 wt. %, about 88 wt. %, about 89 wt. %, or about 90 wt. % zeolite ammonia-ZSM-5 powder (SiO₂/Al₂O₃). In an exemplary embodiment, the uncoated monolithic support structure may comprise about 44 wt. % zeolite ammonia-ZSM-5 powder (SiO₂/Al₂O₃). In a different exemplary embodiment, the uncoated monolithic support structure may comprise about 88 wt. % zeolite ammonia-ZSM-5 powder (SiO₂/Al₂O₃).

(ii) Bentonite Clay

In an embodiment, the uncoated monolithic support structure may comprise from about 1 wt. % to about 20 wt. % bentonite clay. In some embodiments, the uncoated monolithic support structure may comprise from 1 wt. % to about 20 wt. %, about 1 wt. % to about 15 wt. %, or from about 5 wt. % to about 15 wt. % bentonite clay. In other embodiments, the uncoated monolithic support structure may comprise about 1 wt. %, about 5 wt. %, about 10 wt. %, about 15 wt. %, or about 20 wt. % bentonite clay. In an exemplary embodiment, uncoated monolithic support structure may comprise about 10 wt. % bentonite clay.

(iii) Additional Components

In another embodiment, the uncoated monolithic support structure as described herein above may further comprise amorphous silica, a plasticizing binder, a metal dopant, and combinations thereof.

(a) Amorphous Silica

In an embodiment, the uncoated monolithic support structure may comprise from about 1 wt. % to about 95 wt. % amorphous silica. In some embodiments, the uncoated monolithic support structure may comprise from 1 wt. % to about 95 wt. %, from about 10 wt. % to about 95 wt. %, from about 20 wt. % to about 95 wt. %, from about 30 wt. % to about 95 wt. %, from about 40 wt. % to about 90 wt. %, or from about 45 wt. % to about 90 wt. % amorphous silica.

In additional embodiments, the uncoated monolithic support structure may comprise from about 40 wt. % to about 50 wt. % amorphous silica. In other embodiments, the uncoated monolithic support structure may comprise about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, or about 50 wt. % amorphous silica. In additional embodiments, the uncoated monolithic support structure may comprise from about 80 wt. % to about 90 wt. % amorphous silica. In other embodiments, the uncoated monolithic support structure may comprise about 80 wt. %, about 81 wt. %, about 82 wt. %, about 83 wt. %, about 84 wt. %, about 85 wt. %, about 86 wt. %, about 87 wt. %, about 88 wt. %, about 89 wt. %, or about 80 wt. % amorphous silica. In an exemplary embodiment, the uncoated monolithic support structure may comprise about 44 wt. % amorphous silica. In a different exemplary embodiment, the uncoated monolithic support structure may comprise about 88 wt. % amorphous silica.

(b) Plasticizing Binders

Suitable plasticizing binders may include, without limit methyl cellulose and polyvinyl chloride. In an exemplary embodiment, the plasticizing organic binder may be methyl cellulose.

In an embodiment, the uncoated monolithic support structure may comprise from about 1 wt. % to about 10 wt. % plasticizing organic binder. In other embodiments, the uncoated monolithic support structure may comprise from about 1 wt. % to about 10 wt. %, about 1 wt. % to about 8 wt. %, about 1 wt. % to about 6 wt. %, or about 1 wt. % to about 4 wt. %. In some embodiments, the uncoated monolithic support structure may comprise about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, or about 10 wt. % plasticizing organic binder. In an exemplary embodiment, the uncoated monolithic support structure may comprise about 2.5 wt. % plasticizing organic binder.

(c) Metal Dopants

Suitable metal dopants may include, without limit, Zn, Ce, Cr, Mg, Cu, La, Ga, Y, Mo, Ni, and Fe.

In an exemplary embodiment, the metal dopant may be selected from the group consisting of Zn, Ce, Cr, Mg, Cu, La, Ga, Y, and combinations thereof.

In an embodiment, the uncoated monolithic support structure may comprise from about 1 wt. % to about 10 wt. % metal dopant. In other embodiments, the uncoated monolithic support structure may comprise from about 1 wt. % to about 10 wt. %, about 1 wt. % to about 9 wt. %, about 2 wt. % to about 8 wt. %, or about 3 wt. % to about 8 wt. %. In some embodiments, the zeolite monolith may comprise about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, or about 10 wt. % metal dopant. In an exemplary embodiment, the uncoated monolithic support structure may comprise about 4.5 wt. % metal dopant. In a different exemplary embodiment, the uncoated monolithic support structure may comprise about 5.5 wt. % metal dopant.

(iv) Coating

In an embodiment, the uncoated monolithic support structure may be coated to produce a zeolite coated monolith article.

Suitable coatings may include, without limit, SAPO-34, SSZ-13 and UZM-9.

(v) Physical Dimensions

In an embodiment, the zeolite monolith may have a wall thickness of from about 0.2 mm to about 0.9 mm. In some embodiments, the zeolite monolith may have a wall thickness of from about 0.2 mm to about 0.9 mm, about 0.3 mm to about 0.9 mm, about 0.4 mm to about 0.9 mm, about 0.5 mm to about 0.9, about 0.5 mm to about 0.8 mm, or about 0.5 mm to about 0.8 mm. In other embodiments, the zeolite monolith may have a wall thickness of about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75, about 0.8 mm, about 0.85 mm, or about 0.9 mm. In an exemplary embodiment, the zeolite monolith may have a wall thickness of about 0.6 mm.

In an embodiment, the zeolite monolith may have a square channel length of from about 0.2 mm to about 1.6 mm. In some embodiments, the zeolite monolith may have a square channel length of from about 0.2 mm to about 1.6 mm, about 0.3 mm to about 1.6 mm, about 0.4 mm to about 1.6 mm, about 0.5 mm to about 1.6 mm, about 0.6 mm to about 1.6 mm, about 0.7 mm to about 1.6 mm, about 0.8 mm to about 1.6 mm, about 0.9 mm to about 1.6 mm, about 0.9 mm to about 1.5 mm, about 0.9 mm to about 1.4 mm, about 1.0 mm to about 1.4 mm, or about 1.1 mm to about 1.4 mm. In other embodiments, the zeolite monolith may have a square channel length of about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8, about 0.85, about 0.9 mm, about 0.95 mm, about 1.0 mm, about 1.1 mm, about 1.2 mm, about 1.3 mm, about 1.4 mm, about 1.5 mm, or about 1.6 mm. In an exemplary embodiment, the zeolite monolith may have a square channel length of about 1.2 mm.

In an embodiment, the zeolite monolith may have a total pore volume of from about 0.2 cm³/g to about 0.95 cm³/g. In some embodiments, the zeolite monolith may have a total pore volume of about 0.2 cm³/g, about 0.25 cm³/g, about 0.3 cm³/g, about 0.35 cm³/g, about 0.4 cm³/g, about 0.45 cm³/g, about 0.5 cm³/g, about 0.55 cm³/g, about 0.6 cm³/g, about 0.65 cm³/g, about 0.7 cm³/g, about 0.75 cm³/g, about 0.8 cm³/g, about 0.9 cm³/g, or about 0.95 cm³/g.

In an embodiment, the zeolite monolith may have a mesoporosity of from about 0.1 cm³/g to about 0.95 cm³/g. In some embodiments, the zeolite monolith may have a mesoporosity of about 0.1 cm³/g, about 0.15 cm³/g, about 0.2 cm³/g, about 0.25 cm³/g, about 0.3 cm³/g, about 0.35 cm³/g, about 0.4 cm³/g, about 0.45 cm³/g, about 0.5 cm³/g, about 0.55 cm³/g, about 0.6 cm³/g, about 0.65 cm³/g, about 0.7 cm³/g, about 0.75 cm³/g, about 0.8 cm³/g, about 0.9 cm³/g, or about 0.95 cm³/g

### (b) Manufacture

Another aspect of the present disclosure encompasses a method of producing a zeolite monolith article, the comprising (a) calcinating the ammonia-ZSM-5 powder (SiO₂/Al₂O₃) at a temperature from about 500° C. to about 600° C. for about 2 hours to about 8 hours to produce a mixture comprising ZSM-5 zeolite and Y zeolite; (b) mixing the mixture comprising ZSM-5 zeolite and Y zeolite with bentonite clay and water to produce a homogenous slurry; and (c) 3D-printing the homogenous slurry on an alumina substrate to produce the zeolite monolith article.

Additional aspects of the method will be described in further detail below.

(i) Calcination Step

In general, the ammonia-ZSM-5 powder (SiO₂/Al₂O₃=50) is calcinated to produce a mixture comprising ZSM-5 zeolite (HZSM-5) and Y zeolite (SiO₂/Al₂O₃=80). The calcination step may be performed using standard techniques known to those of skill in the art.

In an embodiment, the calcination step may be conducted at a temperature from about 500° C. to about 1100° C. In some embodiments, the calcination step may be conducted at a temperature of about 500° C., about 550° C., about 600° C., about 650° C., about 700° C., about 750° C., about 800° C., about 850° C., about 900° C., about 950° C., about 1000° C., about 1050° C., or about 1100° C. In an exemplary embodiment, the calcination step may be conducted at a temperature of about 550° C.

In an embodiment, the calcination step may be conducted for about 2 hours to about 8 hours. In some embodiments, the calcination step may be conducted for about 2 hours, about 2.5 hours, about 3 hours, about 3.5 hours, about 4 hours, about 4.5 hours, about 5 hours, about 5.5 hours, about 6 hours, or about 6.5 hours

(ii) Mixing Step

In general, the components, as described in Section (I)(a)(i)-(I)(a)(iii), above, of the zeolite monolith article may be combined with water to produce a homogenous slurry using standard techniques known to those of skill in the art.

(iii) 3D Printing Step

In general, the homogenous slurry may be 3D-printed on a substrate to generate the zeolite monolith. 3D-printing techniques are known to those of skill in the art.

In an embodiment, the zeolite monolith article may be printed on an alumina substrate, In an exemplary embodiment, the zeolite monolith may be printed on an alumina substrate.

In an embodiment, the zeolite monolith article may be printed in any shape known to those of skill in the art. In some embodiments, the zeolite monolith is printed in a circular honeycomb structure or a square honeycomb structure.

(iv) Coating Step

The zeolite monolith article may be coated using techniques known to those of skill in the art, for example, see Li, X., Chemical Engineering Journal, 333 (2018) 545-553, which is hereby incorporated by reference in its entirety. Briefly, the 3D printed zeolite monolith article is immersed in a water suspension comprising from about 0.1 wt. % to about 2 wt. % seed particles of a coating. The seeded zeolite monolith article is then subjected to hydrothermal treatment process to grow the coating to produce the coated zeolite monolith.

SAPO-34 may be prepared according to known procedures, for example, see Li, X., Chemical Engineering Journal, 333 (2018) 545-553, which is hereby incorporated by reference in its entirety. Briefly, aluminum isopropoxide (Al(i-C₃H₇O)₃, colloidal silica (40 wt. % SNOWTEX-ZL), tetraethylammonium hydroxide (TEAOH, 40 wt. %), and H₃PO₄ (85 wt. %) are mixed together with a molar ratio of about 0.5 to about 1.5 Al₂O₃: about 0.5 to about 1.5 P₂O₅: about 0.1 to about 1.0 SiO₂: about 1.0 to about 10.0 TEACH: about 100 to about 180H₂O. The SAPO-34 mixture may then be subjected to a hydrothermal treatment process. Recovering SAPO-34 seeds involves centrifuging, washing, and drying.

The hydrothermal treatment process may be conducted at a temperature of from about 150° C. to about 250° C. In some embodiments, the hydrothermal treatment process is conducted at about 150° C., about 160° C., about 170° C., about 180° C., about 190° C., about 200° C., about 210° C., about 220° C., about 230° C., about 240° C., or about 250° C. In an exemplary embodiment, the hydrothermal process may be conducted at about 180° C. In a different exemplary embodiment, the hydrothermal process may be conducted at about 220° C.

The hydrothermal process may be conducted for about 1 hour to about 8 hours. In some embodiments, the hydrothermal process may be conducted for about 1 hour, about 1.5 hours, about 2 hours, about 2.5 hours, about 3 hours, about 3.5 hours, about 4 hours, about 4.5 hours, about 5 hours, about 5.5 hours, or about 6 hours.

### (II) Methods of Use

### (a) Conversion of Methanol to Light Olefins

Another aspect of the present disclosure encompasses a process for methanol to one or more light olefins (MTO), the process comprising contacting methanol, under deoxygenation conditions, with a catalyst comprising a zeolite monolith, wherein the zeolite monolith comprises an uncoated monolithic support structure including walls having a honeycomb structure comprising ammonia-ZSM-5 powder (SiO₂/Al₂O₃) and bentonite clay, and a porous coating disposed directly upon the uncoated monolithic support structure.

The zeolite monolith is described in greater detail in Section (I) hereinabove.

In general, the MTO process is known to those of skill in the art.

(i) Light Olefins

Suitable light olefins include, without limit, ethylene, propylene, and butylene. In some embodiments, the light olefin is selected from the group consisting of ethylene, propylene, butylene, and combinations thereof.

(ii) Conditions

In an embodiment, the MTO process occurs at a temperature of from about 500° C. to about 1500° C. In some embodiments, MTO process occurs at a temperature of about 500° C., about 525° C., about 550° C., about 575° C., about 600° C., about 625° C., about 650° C., about 675° C., about 700° C., about 725° C., about 750° C., about 775° C., about 800° C., about 825° C., about 850° C., about 875° C., about 900° C., about 925° C., about 950° C., about 975° C., about 1000° C., about 1025° C., about 1050° C., about 1075° C., about 1100° C., about 1125° C., about 1150° C., about 1175° C., about 1200° C., about 1225° C., about 1250° C., about 1275° C., about 1300° C., about 1325° C., about 1350° C., about 1375° C., about 1400° C., about 1425° C., about 1450° C., about 1475° C., or about 1500° C.

(iii) Selectivity

In an embodiment, the MTO process has a selectivity towards ethylene and propylene.

(iv) Reactor

In an embodiment, the conversion of methanol to one or more light olefins (MTO) process may occur in a tubular reactor.

In an embodiment, the reactor is a fixed bed reactor or a fluidized-bed reactor. In an exemplary embodiment, the reactor is a fixed bed reactor.

### (b) Hydrocarbon Cracking

An additional aspect of the present disclosure encompasses a method for catalytic cracking a hydrocarbon to produce a light olefin, the process comprises contacting the hydrocarbon, under cracking conditions, with a catalyst comprising a zeolite monolith, wherein the zeolite monolith comprises an uncoated monolithic support structure including walls having a honeycomb structure comprising ammonia-ZSM-5 powder (SiO₂/Al₂O₃) and bentonite clay; and a porous coating disposed directly upon the uncoated monolithic support structure.

The zeolite monolith is described in greater detail in Section (I) hereinabove.

In general, the cracking process is known to those of skill in the art.

(i) Hydrocarbons

In an embodiment, the hydrocarbon may be a light alkane.

Suitable light alkanes include, without limit, ethane, propane, butane, pentane, and hexane. In some embodiments, the light alkane is a hexane. In an exemplary embodiment, the light alkane is n-hexane.

(ii) Light Olefins

Suitable light olefins include, without limit, ethylene, propylene, and butylene. In some embodiments, the light olefin is selected from the group consisting of ethylene, propylene, butylene, and combinations thereof.

(iii) Cracking Conditions

In an embodiment, the catalytic cracking occurs at a temperature of from about 550° C. to about 700° C. In some embodiments, the catalytic cracking occurs at a temperature of about 550° C., about 575° C., about 600° C., about 625° C., about 650° C., about 675° C., or about 700° C.

In an embodiment, the catalytic cracking occurs at a pressure of from about 0.5 bar to about 2 bar. In some embodiments, the catalytic cracking occurs at a pressure of about 0.5 bar, about 0.75 bar, about 1.0 bar, about 1.25 bar, about 1.5 bar, about 1.75 bar, or about 2.0 bar.

(iv) Reactor

In an embodiment, the catalytic cracking process may occur in a tubular reactor.

In an embodiment, the reactor is a fixed bed reactor or a fluidized-bed reactor.

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated, For purposes of this invention, the chemical elements are identified in accordance with the Periodic Table of the Elements, CAS version, and the Handbook of Chemistry and Physics, 75^{th} Ed, 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M, B. and March, J., eds. John Wiley & Sons, New York: 2001, the entire contents of which are hereby incorporated by reference.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following abbreviations are used throughout the Examples: XRD: x-ray crystallography; SEM: scanning electron microscopy; FTIR: Fourier-transform infrared spectroscopy; NH₃-TPD/R: ammonia-temperature programmed desorption/resorption; and MAS NMR: Magic angle spinning (MAS) nuclear magnetic resonance (NMR).

### Example 1: Metal-Doped 3D Printed Zeolite Catalyst with High Resistance to Coke Formation for Methanol Deoxygenation

### Introduction

As one of the most significant reaction in C1 chemistry, the methanol-to-olefins (MTO) reaction, provides an alternative approach for producing basic petrochemicals from non-oil resources such as coal and nature gas.¹⁻³ Driven by the increasing demand for ethylene and propylene, which are the primary building blocks for the polymer industry,⁴ this process can be a readily implemented by current technologies via synthesis gas, natural gas, biomass and the coal.⁵⁻⁹ MTO reaction is mostly catalyzed on acidic zeolite catalysts. With ZSM-5 and SAPO-34 being the central focus due to their distinct selectivity in generating light olefins.^{10, 11} Å high rate of deactivation is usually observed for SAPO-34, which possesses CHA framework with small channels and cages, due to the rapid coke deposition.^{12, 13} Therefore, ZSM-5 zeolite catalysts are more often compromisingly used despite lower olefin yield.¹⁴⁻¹⁷

Various strategies have been employed to increase the selectivity towards light olefins over ZSM-5 zeolite with MFI framework by optimizing the acidity,¹⁸⁻²⁰ scaling down the crystal size,^{15, 21, 22} altering the pore structure,²³⁻²⁵ and modifying with heteroatoms.²⁶⁻²⁸ Essentially, introducing heteroatoms in the zeolite framework modifies the acidity of the catalysts. Efforts have been made to dope ZSM-5 with various metals (including alkali metal,²⁹ alkaline earth metal,³⁰ and transition metal³¹), nonmetals (mainly phosphorus),³² and semimetals (mainly boron)³³. A facile method to dope ZSM-5 with metals is to incorporate the element into the framework of the zeolite. In the synthesis step, the aluminum atoms in the MFI framework are substituted with atoms such as B, Ga, and Fe and the product is generally known as isomorphously substituted ZSM-5.^{34, 35} ZSM-5 can also be modified by adding protons and extra-framework cations (mainly metal) to form acid/base or redox sites as a post-treatment step. The most common methods for doping the zeolite with metals are ion-exchange and impregnation techniques.³⁶ However, the preparation of catalysts via these techniques remains quite complex and costly due to the low controllability at micro-scale and sensitivity to pH value of zeolite crystals in the cation solution.

Recent developments in three-dimensional (3D) printing of various porous materials, such as zeolites,^{37, 38} silicoaluminophosphate,³⁹ aminosilica,⁴⁰ and metal-organic frameworks,⁴¹ make it possible to efficiently prepare novel materials with tunable structural, physicochemical and mechanical properties for broad applications. Our recent work³⁸ demonstrated the feasibility and the advantages of preparing 3D-printed zeolite monolith as a promising catalyst for alkane cracking. The 3D-printed ZSM-5 monoliths showed promoted stability and increased selectivity towards light olefins in n-hexane cracking as a result of the formation of hierarchical pores and moderate acidity. In another investigation Tubío and coworkers⁴² synthesized Cu/Al₂O₃ catalytic system with a woodpile porous structure using 3D printing technique. It was proclaimed that active component (Cu) was immobilized in the Al₂O₃ matrix and the leaching of the metal into the reaction medium was avoided. The 3D-printed catalyst also showed good dispersion of the copper and excellent performance in Ullmann reaction.

Numerous metals have been employed as promoters in ZSM-5 type zeolite for the conversion of methanol to hydrocarbons and the effect of promoter on the product distribution has been shown to vary from metal to metal. Hadi et al.⁴³ reported that Ce is a promising promoter for Mn/H-ZMS-5 in the process of methanol conversion to propylene. The selectivity towards propylene was dramatically enhanced and the propylene/ethylene ratio was increased. Several catalysts comprising zeolite ZSM-5 impregnated with Cu were tested for methanol to hydrocarbons by Conte and coworkers⁴⁴ and it was found that Cu/ZSM-5 was selective for C₉-C₁₁ aromatic products owing to the interaction of the acid sites of the zeolite with the basic sites of the metal oxide at the edge of the zeolite crystals. In another study by Li and coworkers,⁴⁵ Cu/ZSM-5 prepared via post-treatment method showed improved catalyst lifetime in methanol conversion reaction. Presented and supported by the work of Bakare et al.,⁴⁶ Mg modified ZSM-5 exhibited the most stable activity in the MTO reaction with the highest selectivity to propylene due to the presence of weak Brønsted acid sites. The promoted aromatics production by Zn modified ZSM-5 in methanol conversion was claimed and demonstrated by Xu and coworkers.⁴⁷ Furthermore, metals such as Cr, La, and Y were also introduced to MFI zeolite as promoters for the production of light olefins via catalytic cracking of various alkanes or the dehydration of ethanol.⁴⁸⁻⁵⁰

In this study, recently reported 3D-printed zeolite monolith studes³⁸ were extended to metal-doped zeolite monoliths by introducing various metal dopants via the addition of the precursor into the synthesis paste, aiming at modifying the zeolite monolith properties for enhanced catalytic performance in MTO processes. Guided by the above-mentioned literature, eight metals namely cerium, chromium, copper, gallium, lanthanum, magnesium, yttrium, and zinc were selected for this investigation. The metal-doped 3D-printed monoliths, along with their bare counterpart, were analyzed using various characterization techniques including XRD, SEM, N₂ physisorption, FTIR, and NH₃-TPD/R. These novel materials were tested in the methanol conversion and the effect of each metal dopant on the product distribution was discussed.

### Experimental

Preparation of 3D-Printed M/ZMS-5 Monoliths:

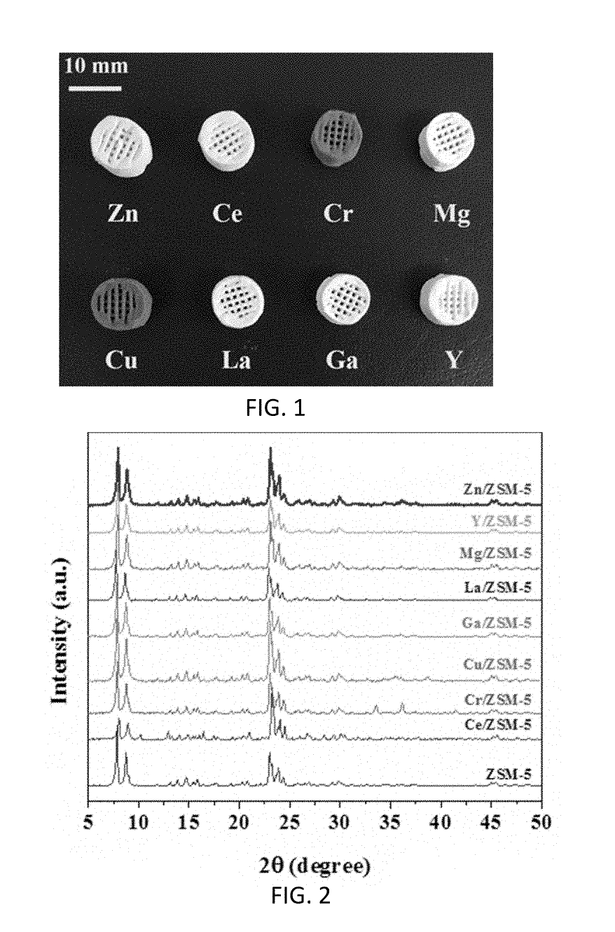

The bare ZSM-5 monoliths were prepared using the method reported in our previous work.³⁸ The 3D-printed metal-doped zeolite monoliths were prepared by adding metal precursor solution into the zeolite and bentonite clay mixture while making the paste. The metal precursor used were Ce(NO₃)₃.6H₂O, Cr(NO₃)₃.9H₂O, Cu(NO₃)₂.2.5H₂O, Ga(NO₃)₃.xH₂O, La(NO₃)₃.xH₂O, Mg(NO₃)₂.6H₂O, Y(NO₃)₃.6H₂O and Zn(NO₃)₂.6H₂O purchased from Sigma-Aldrich (St. Louis, Mo.). About 8 wt. % metal precursor were added into the paste and the paste was extruded using our scale printed, as described in our early work.³⁸ All the fresh 3D-printed monoliths were calcined at 823 K for 6 hours in order to decompose and remove the methyl cellulose, enhance the mechanical strengthen and immobilize the metal atoms. Bare HZSM-5 monolith is denoted as “ZSM-5” while the samples with metal dopants are denoted as M/ZSM-5 (M=Ce, Cr, Cu. Ga, La, Mg, Y or Zn). All the as-prepared M/ZSM-5 monoliths with diameter of 10 mm are shown in FIG. 1.

Characterizations of the 3D-Printed M/ZSM-5 Monoliths:

X-ray diffraction (XRD) patterns were recorded on a PANalytical X'Pert multipurpose X-ray diffractometer in the angle (28) range of 5° to 50° with Cu-Kα1 radiation (40 kV and 40 mA) at a rate of 2.0° min⁻¹. Nitrogen physisorption measurements were performed on a Micromeritics 3Flex surface characterization analyzer at 77 K. Prior to the measurements, all samples were degassed at 573 K for 6 hours. Total surface area was determined by the Brunauer-Emmett-Teller (BET) equation using the relative pressure (P/P₀) in the range of 0.05-0.3. External surface area was calculated using t-plot method and the pore size distribution was estimated using Barrett-Joyner-Halenda (BJH) model. Scanning electron microscopy (SEM) images were captured on a Hitachi S-4700 instrument to investigate the morphology of the materials. Energy-dispersive X-ray spectroscopy (EDS) was carried out to map the presence of various elements in the doped zeolite monoliths. Temperature-programmed desorption of ammonia (NH₃-TPD) was performed to investigate the acid property of the samples. NH₃ adsorption was carried out on the Micromeritics 3Flex analyzer under a flow of 5 vol. % NH₃/He at 373 K. The desorption of NH₃ was measured from 373 to 873 K at a constant heating rate of 10 K min⁻¹. A mass spectroscopy (BELMass) was used to detect the quantity of NH₃ desorption. Temperature-programmed Reduction with hydrogen (H₂-TPR) was also performed from 323 to 1123 K under a flow of 5 vol % H₂/He using the same instrument. To determine the functional groups, FTIR spectra were obtained using a Nicolet-FTIR Model 750 spectrometer. Mechanical testing was also carried out to determine the mechanical integrity of the monoliths using an Instron 3369 (Instron, Norwood, Mass., USA) mechanical testing device with a 500 N load at 2.5 mm/min. Prior to testing, monoliths were polished with the sandpaper to prevent the uncertain surface and to avoid cracks on the surface for achieving effective results. Compressive force was applied until the monolith broke. Thermogravimetric analysis-differential thermal analysis (TGA-DTA) of the spent catalysts was carried out from 303 K to 1173 K using TGA (Model Q500, TA Instruments), at a rate of 10 K/min in a 60 mL min⁻¹ air flow.

Catalytic Test:

Catalytic behavior of the 3D-printed monoliths was assessed in a fixed-bed reactor setup. Nitrogen flow saturated with methanol at 303 K was fed to the stainless steel reactor. The feed flow rate was controlled by a mass flow controller (Brooks, 5850). In a typical run, 0.3 g of catalyst was tested under 673 K at 1.01 bar with a weight hourly space velocity (WHSV) of 0.35 h⁻¹. The catalyst was activated in situ at 823 K in nitrogen flow for 2 hours. The products were directly transferred to an on-line gas chromatography (SRI 8610C) and analyzed every hour with a flame ionized detector (GC-FID) connected to mxt-wax/mxt-alumina capillary column. The inlet line to the reactor was kept heated at 383 K whereas the effluent line of the reactor until GC injector was kept at 418 K to avoid potential condensation of hydrocarbons.

### Results and Discussion

Characterization of the 3D-Printed M/ZSM-5 Monoliths:

The XRD patterns of the 3D-printed ZMS-5 monolith and M/ZSM-5 monoliths are depicted in FIG. 2. All the doped monoliths exhibited the typical diffraction peaks of MFI structure at around 2θ=8.0°, 9.0°, 14.8°, 22.9°, 24.0°, and 29.8° corresponding to (101), (200), (301), (501), (303), and (503) planes respectively,⁵¹ which indicated that the zeolite framework was retained after the employment of all the investigated metal dopants. Moreover, additional peaks were found in Ce/ZSM-5 pattern at 2θ=28.2° indicating the formation of CeO₂,⁵² in Cr/ZSM-5 pattern at 2θ=33.6°, 36.1°, and 41.5° implying the formation of Cr₂O₃,⁵³ whereas the peaks at 2θ=35.6° and 38.7° in the XRD pattern of Cu/ZSM-5 confirms the formation of CuO.⁵⁴ No additional peaks were observed in other metal-doped samples including Ga, La, Mg, Y, and Zn, which means these metals were highly dispersed in the ZSM-5 particles.⁵⁵

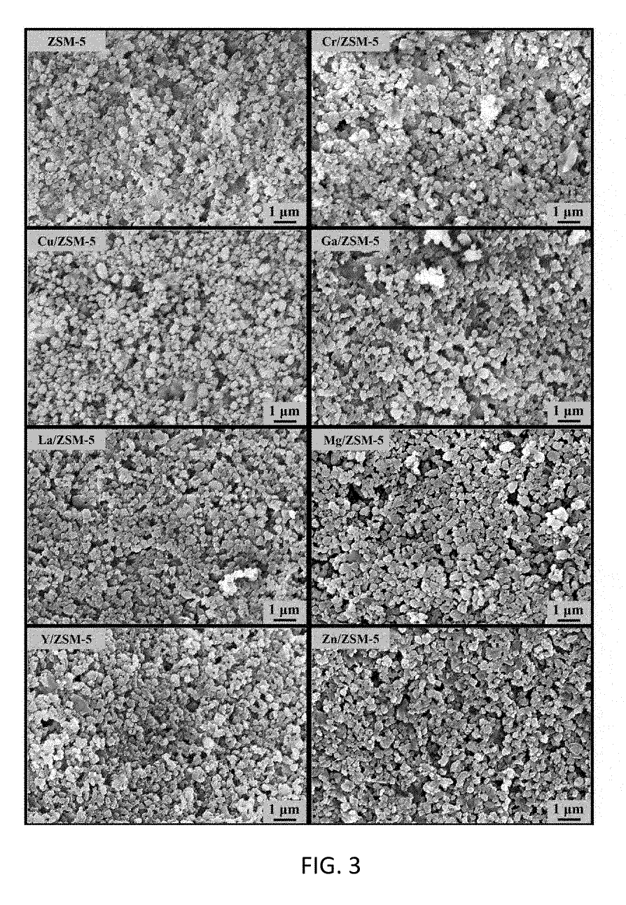

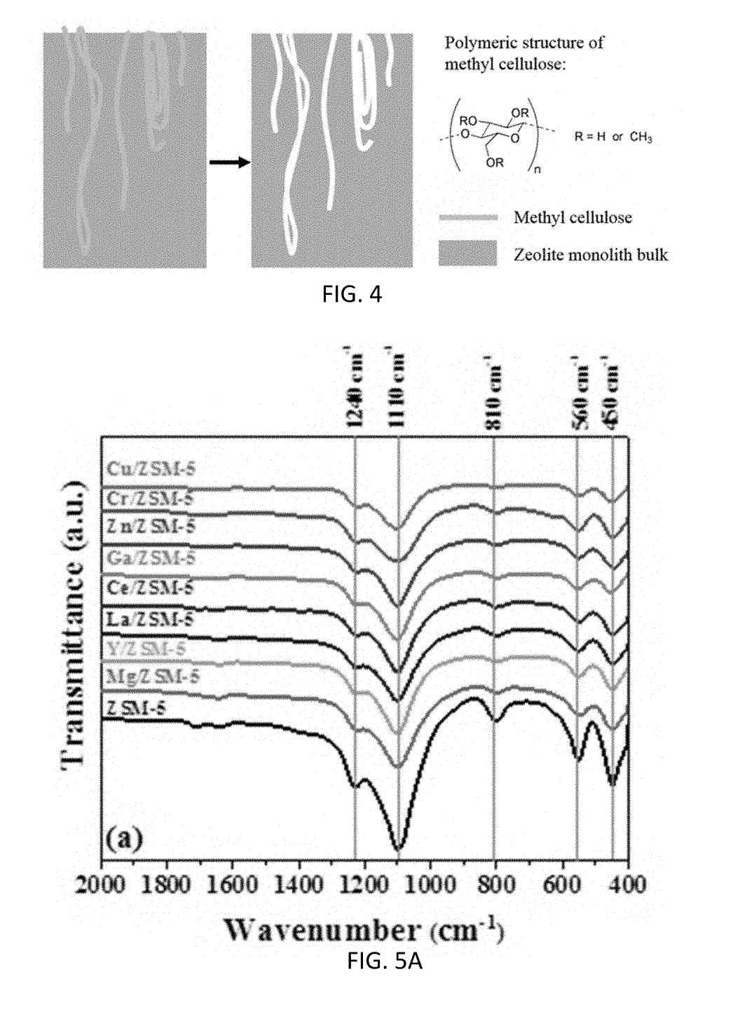

FIG. 3 illustrates the morphology of the ZSM-5 and M/ZSM-5 monolith samples. It is clear that all samples were composed of coffin-like ZSM-5 particles. The particles were well bound due to the addition of bentonite clay, which rendered the monoliths self-standing with structural integrity. As can be observed, pores with a broad range of sizes and irregular shapes were abundantly dispersed on the monolith surface for all doped samples. The formation of the pores are believed to stem from the decomposition of methyl cellulose via calcination.^{37, 56} The polymeric structure of methyl cellulose renders its molecules exist in various sizes according to the changing degree of polymerization. Moreover, the vigorous stir while making the paste may lead to the folding and twisting of the long chains of methyl cellulose. Therefore, pores with a wide size distribution and a variety of shapes were generated, as schematically depicted in FIG. 4.

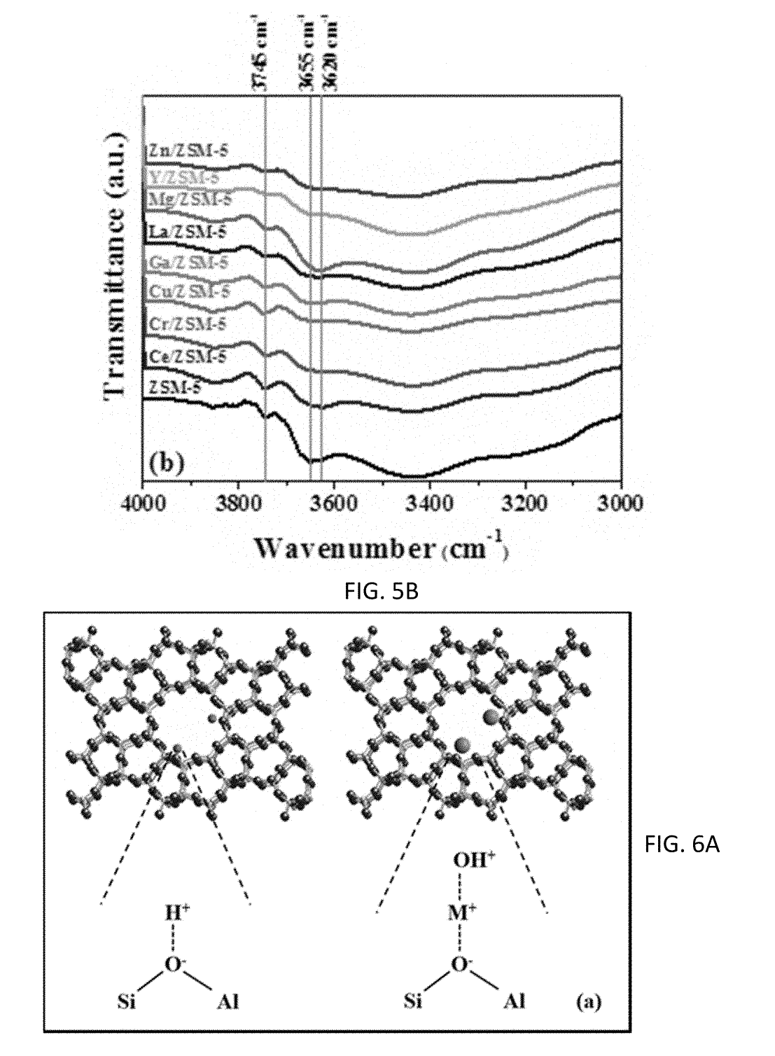

The FTIR spectra of the 3D-printed ZSM-5 monolith and its metal-doped counterparts in the range of 400-2000 cm⁻¹ are presented in FIG. 5A. The absorption bands at 450, 560, 810, 1110, and 1240 cm⁻¹, recognized in all samples, are typical vibrations characteristics of MFI type zeolites.⁵⁷ Specifically, the peak at about 450 cm⁻¹ is associated to the vibration of internal bonds (T-O) of SiO₄ and AlO₄ tetrahedra.⁵⁸ The band observed at 560 cm⁻¹ was ascribed to external bonds of double five-member ring.⁵⁹ The bands locates at around 810 cm⁻¹ corresponded to symmetric stretching of external bonds between tetrahedral.⁸⁹ The strongest absorption peak appeared about 1110 cm⁻¹ was related to the internal asymmetric stretching of Si—O-T bonds.⁶¹ The peak found at 1240 cm⁻¹ was a reflection of the asymmetric stretch vibration of T-O bond assigned to the external linkages between TO₄ tetraheda. The presence of these peaks indicates that the MFI structure remained intact in all the as-prepared metal-doped monoliths, which is in accordance with XRD results.

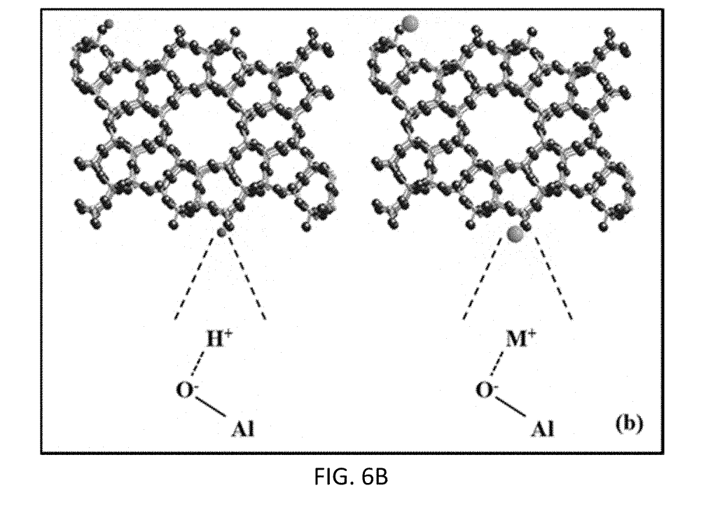

FIG. 5B shows the FTIR spectra with bands in the range of 4000-3000 cm⁻¹, known as the hydroxyl group region. The peak appeared at 3745 cm⁻¹ was ascribed to the —OH vibration of the silanol groups (Si—OH), mostly on the external surface of the zeolite.²⁶ All the investigated metals barely changed this peak, suggesting these metals had little effect on the silanol groups. The peaks ranging from 3665 cm⁻¹ to 3610 cm⁻¹ are generally regarded as the reflection of Al-bonded hydroxyl groups. In detail, the band at 3655 cm⁻¹ was ascribed to the external Al-bonded hydroxyl groups with weak Brønsted acidity while the band at 3620 cm⁻¹ was related to the internal bridging hydroxyl groups (Si—OH—Al) with strong Brønsted acidity.²⁵ It is noteworthy that the bands of M/ZSM-5 (except for Mg/ZSM-5) in this range were much weaker than the bare ZSM-5, indicating the elimination of these two groups by the introduction of metal atom. For Mg/ZSM-5, a strong peak at about 3660 cm⁻¹ was observed. Previous report⁶² attributed this band to the substitution interaction between Mg²⁺ and protons of Si—OH—Al groups to form Mg(OH)⁺, as shown in FIG. 6A. Since the bridging hydroxyl groups generally exist in the internal cages of the zeolite, the metal dopant would be immobilized in the micropores. On the other hand, when the metal exchanges the hydrogen on Al—OH hydroxyl group, which is usually the terminal of the framework and, this metal would locate outside of the framework channels and cages, as shown in FIG. 6B.

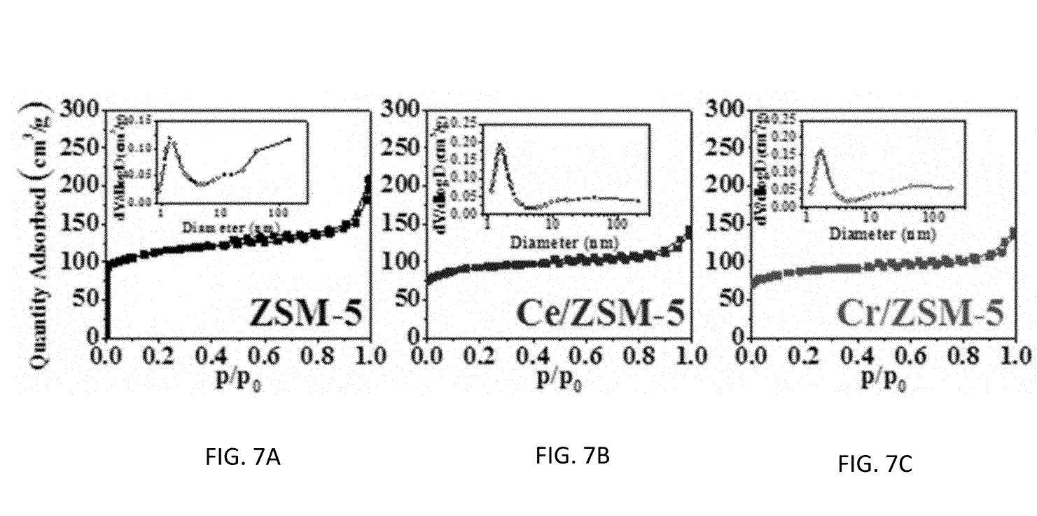

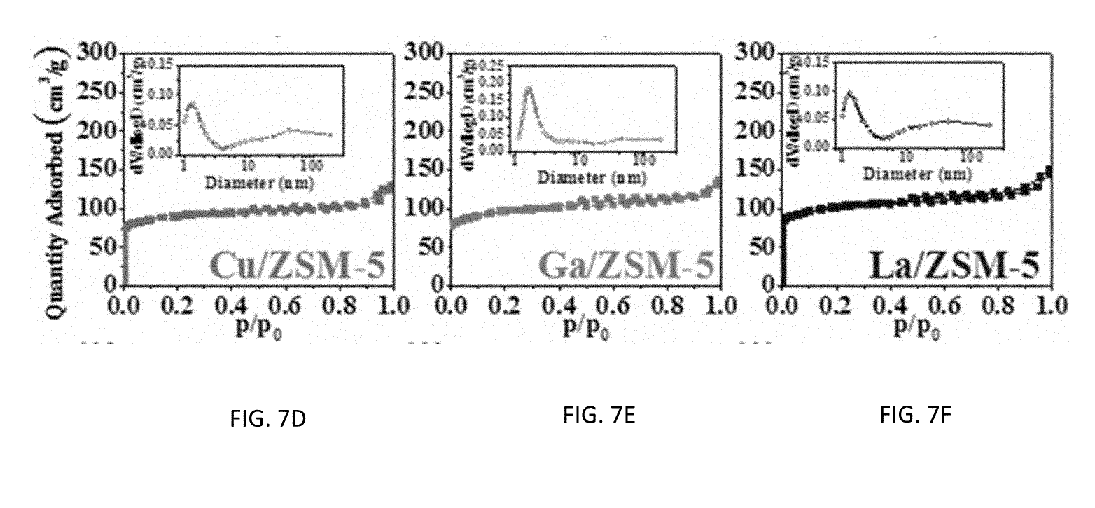

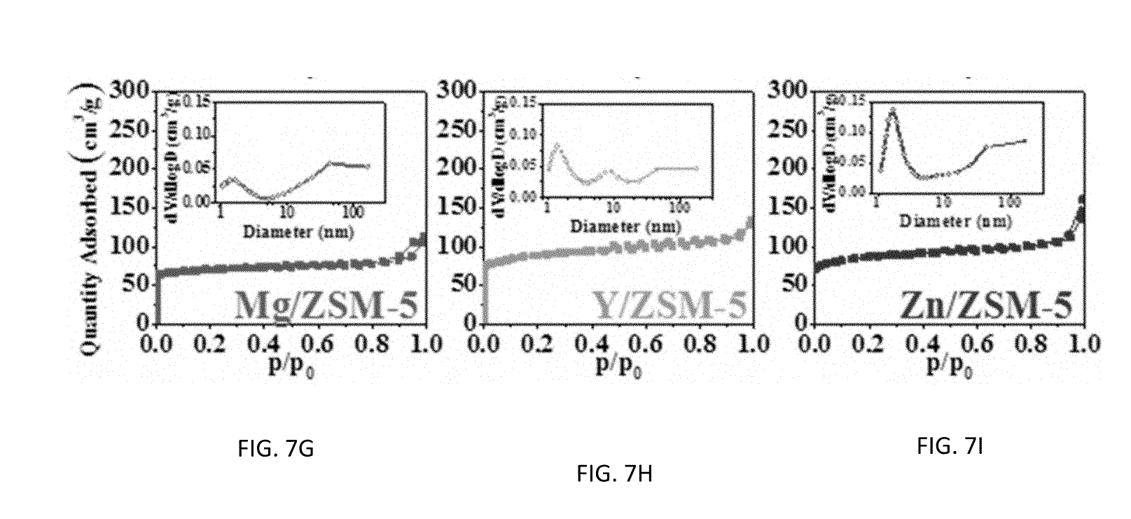

N₂ physisorption isotherms of the as-prepared samples are depicted in FIG. 7, with corresponding pore size distributions shown as inset figures. All isotherms exhibited the combination of type I and type IV isotherm with a significant enhanced uptake in the P/P₀ range of c.a. 0.9-1.00. The hysteresis loop, associated with capillary condensation, indicated the formation of mesoporous structure in all the 3D-printed monoliths.^{63, 64} The pore size distributions curves were estimated by the BJH method using the adsorption branch. For all samples, the first peak of all the samples appeared in the range from 0.5 to 2 nm, which was assigned to the micropores. A slope after around 5 nm can be observed and, thereafter, either a monotonic increase or a broad peak can be observed, suggesting the formation of pores of a wide size distribution, as discussed above.

Table 1 summarizes the total surface area, micopore surface area, external surface area, pore volume, and micropore volume derived from different methods. For comparison, the pristine HZSM-5 powder was also measured and the values of which are listed after the notation of ZSM-5_P while its bare monolith counterpart is noted as ZSM-5_M in the table. The surface areas of the bare ZSM-5 powder and monolith were 429 and 373 cm² g⁻¹ respectively, suggesting the formulation into monolith reduced the total surface area. This might result from the addition of binder and further calcination of fresh monoliths. However, mesopore volume increased from 0.170 cm³ g⁻¹ to 0.200 cm³ g⁻¹, due to the decomposition of the plasticizer. All the investigated metal have effect on the textural properties of the zeolite monolith. Both surface area and pore volume were reduced by metal dopant and the significance of the effect varied from metal to metal. The micropore volume of Ce-, Cu-, Ga-, Y-, and Zn-doped monoliths were found to be 0.096 cm³ g⁻¹, 0.096 cm³ g⁻¹, 0.096 cm³ g⁻¹, 0.090 cm³ g⁻¹, and 0.090 cm³ g⁻¹ respectively, within 10% variation from the bare ZSM-5 monoliths with 0.100 cm³ g⁻¹. It suggests these metals barely entered the micorpores of the zeolite when they were doped in the monolith and they affected the mesopores. Furthermore, Cr- and Mg-doped ZSM-5 monoliths displayed significant decrease in both micopore and mesopore volumes suggesting the existence of the metal dopants in the micropores in addition to mesopores, especially the outstanding low pore volume of Mg/ZSM-5 sample verified the explanation of the FTIR results.

TABLE 1
Physical properties of the investigated samples
obtained from nitrogen physisorption.

| | S_{BET}^{a} | S_{micro}^{b} | Sₑₓₜ | Vₜₒₜₐₗ^{c} | V_{micro} | Vₘₑₛₒ |
|---|---|---|---|---|---|---|
| samples | (m²g⁻¹) | (m²g⁻¹) | (m²g⁻¹) | (cm³g⁻¹) | (cm³g⁻¹) | (cm³g⁻¹) |
| ZSM-5_P | 429 | 261 | 168 | 0.300 | 0.130 | 0.170 |
| ZSM-5_M | 373 | 214 | 159 | 0.300 | 0.100 | 0.200 |
| Ce/ZSM-5 | 303 | 193 | 110 | 0.222 | 0.096 | 0.126 |
| Cr/ZSM-5 | 286 | 180 | 106 | 0.219 | 0.089 | 0.130 |
| Cu/ZSM-5 | 297 | 197 | 100 | 0.202 | 0.096 | 0.106 |
| Ga/ZSM-5 | 318 | 197 | 121 | 0.213 | 0.096 | 0.117 |
| La/ZSM-5 | 335 | 215 | 120 | 0.234 | 0.105 | 0.129 |
| Mg/ZSM-5 | 229 | 178 | 51 | 0.177 | 0.087 | 0.090 |
| Y/ZSM-5 | 293 | 185 | 108 | 0.208 | 0.090 | 0.118 |
| Zn/ZSM-5 | 285 | 185 | 100 | 0.227 | 0.090 | 0.137 |

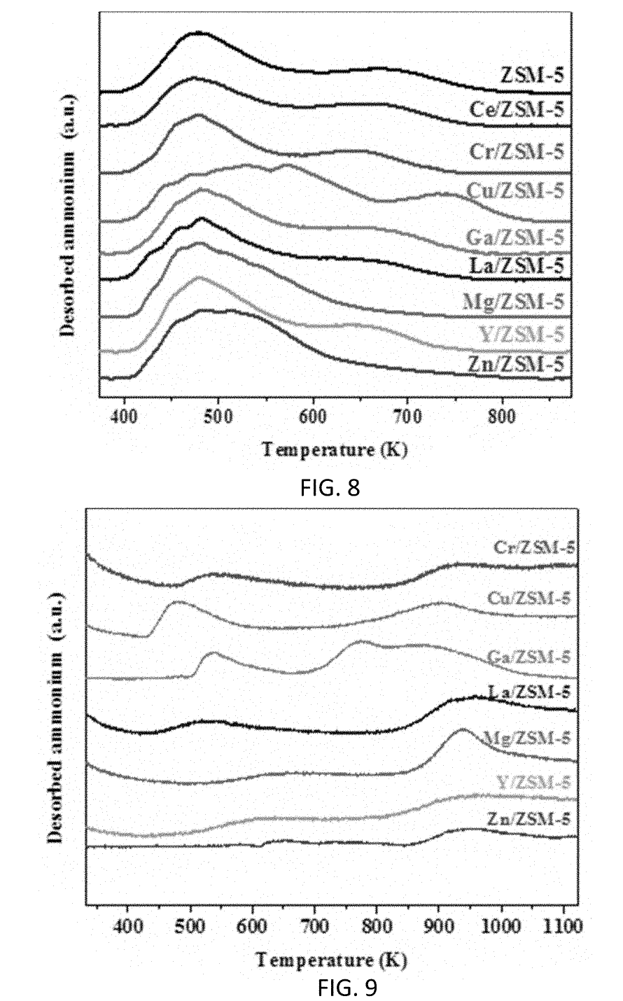

FIG. 8 shows the NH₃-TPD profiles of the 3D-printed monolith samples. For bare ZSM-5 monolith, the peak observed at around 487 K was attributed to the NH₃ desorbed from the weak acid sites (Si—OH, extra framework Al—OH) whereas the peak at 615 K was assigned to the NH₃ desorbed from the strong Brønsted acid sites (mainly located at zeolite inner channels).⁶⁵ As for the metal doped samples, Ce/Cr/Cu/Ga/La/and Y/ZSM-5 monoliths displayed similar desorption profiles in which both weak and strong acid sites were retained after doping with metal but with the reduced intensity, especially for the strong acid sites. The peak of the Cu/ZSM-5 shifts to higher temperature while the rest of dopants in this group shifted to lower temperature. Mg/ZSM-5 and Zn-ZSM-5 exhibited different profiles with only the weak peak and the total amount of weak acid site were obviously increased, suggesting the metal doping converted some of the strong acid sites to the weak sites. The extraordinary acidic property of Mg/ZSM-5, again, highlights its outstanding role as a direct dopant in ZSM-5 monoliths among other metals. The elimination of strong acid sites was in accordance with the proposal from FTIR as well as pore volume results.

H₂-TPR profiles of the M/ZSM-5 samples are presented in FIG. 9. All the metal-doped monoliths displayed H₂ reduction peak but the peak number and position varied from sample to sample due to the difference in reducibility of the metal oxides in zeolite. Unlike other samples with multiple peaks, Mg/ZSM-5 only showed one prime peak at around 938 K owing to the uniformity of the Mg phase existed in the zeolite.

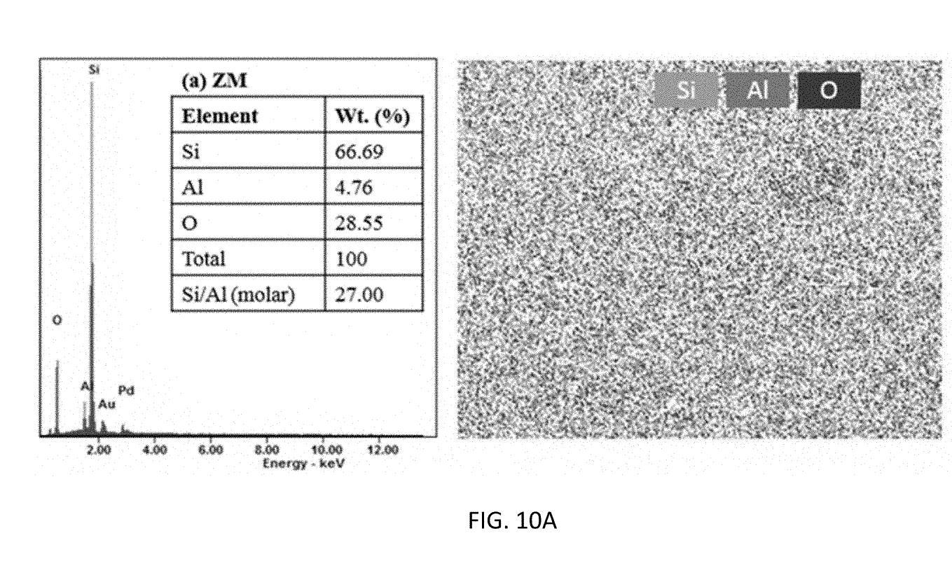

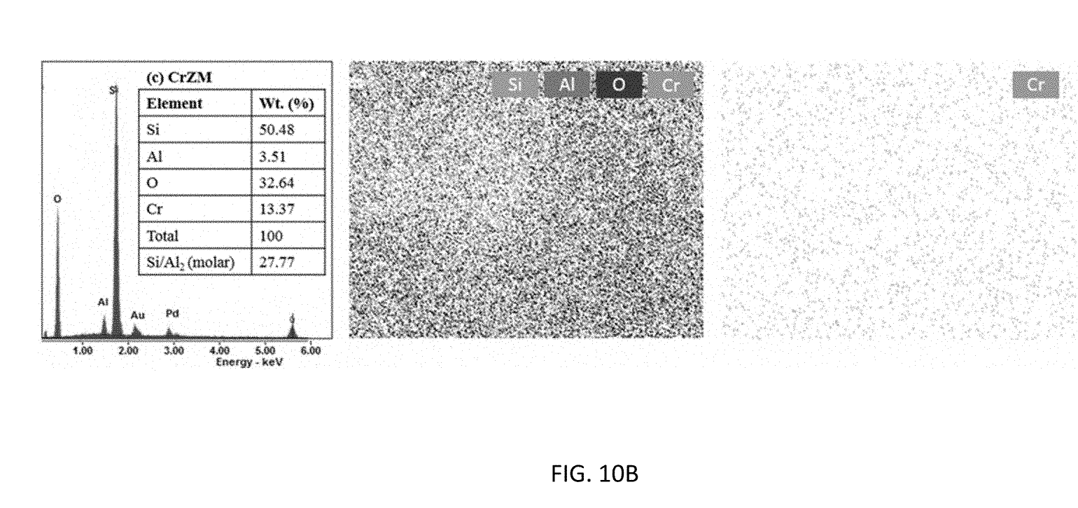

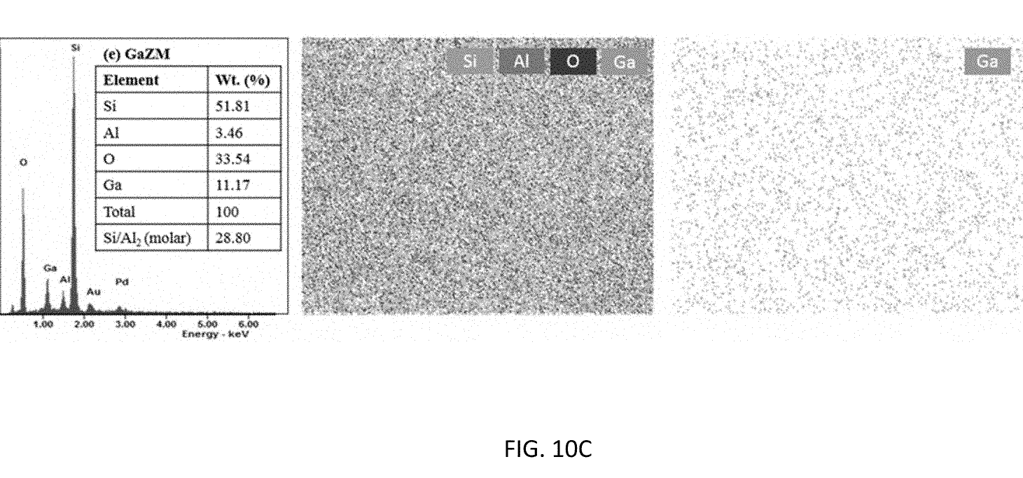

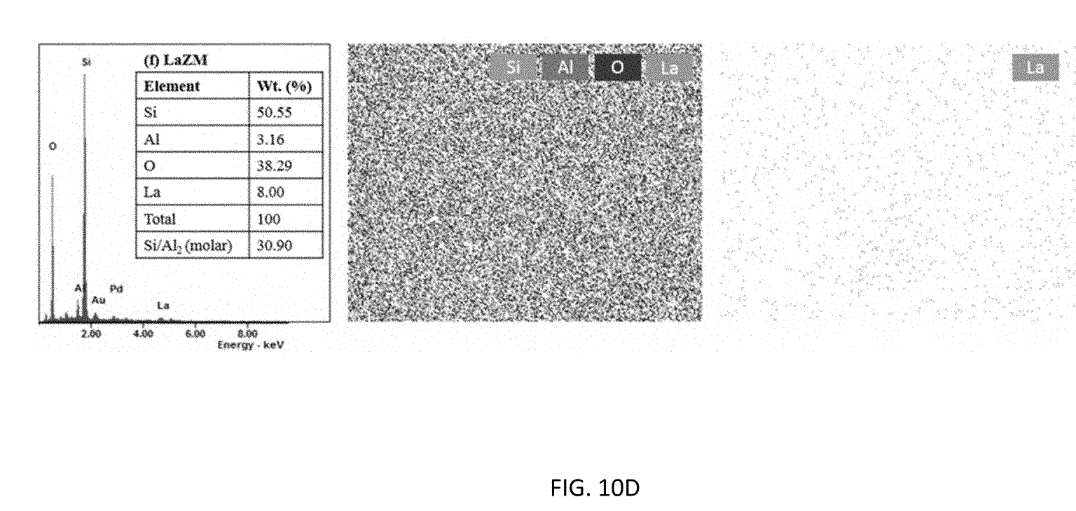

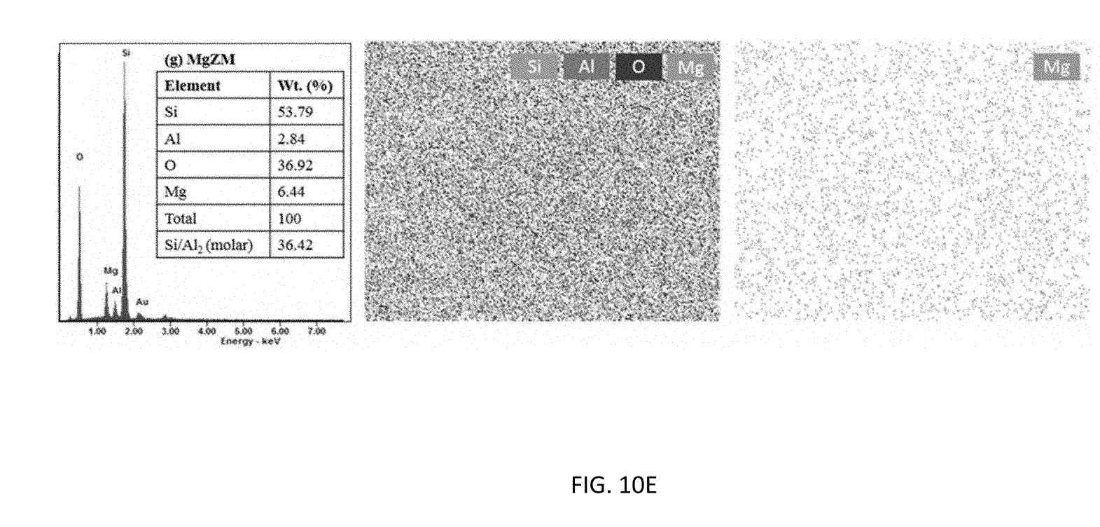

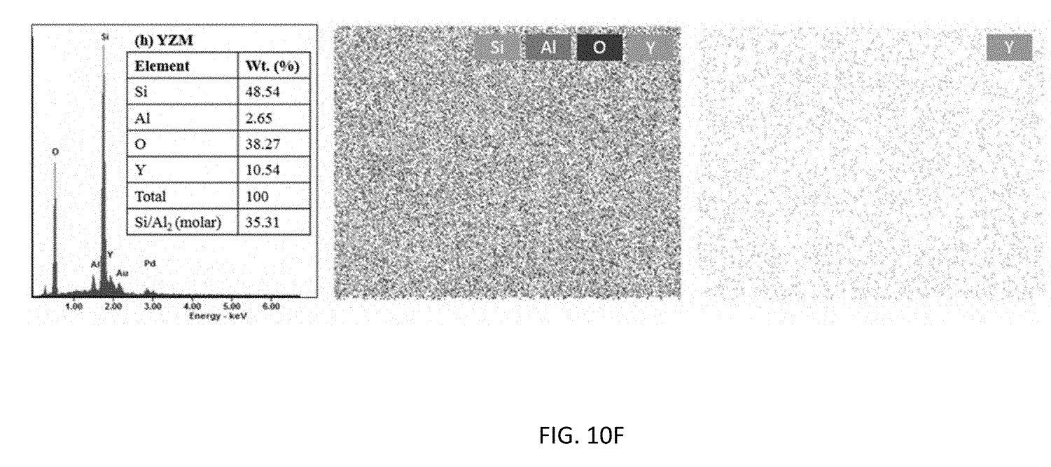

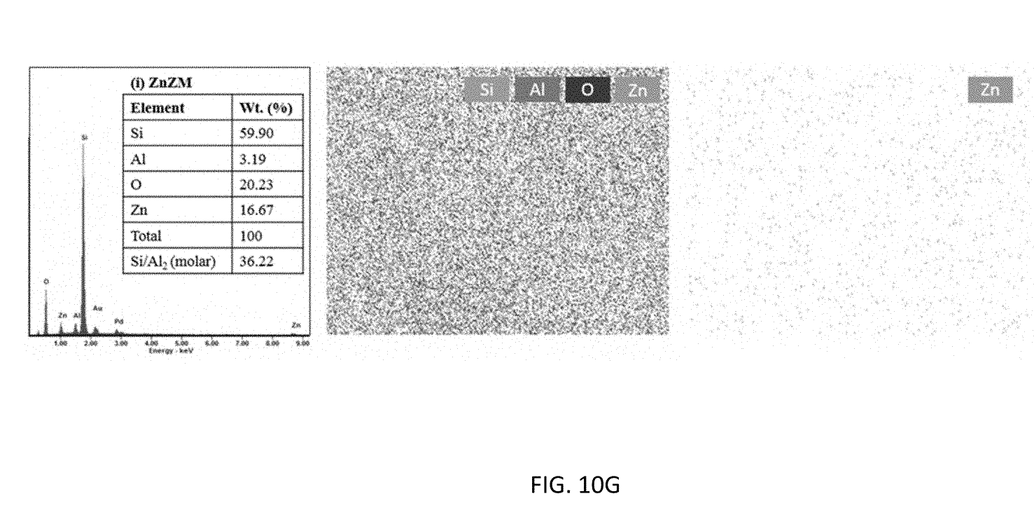

FIG. 10 displayed the mappings of each element in various 3D-printed monolith obtained from EDS. The comparison of the Si/Al₂ ratio between bare ZSM-5 monolith and M/ZSM-5 suggests all dopants caused increases in the Si/Al₂ ratio to various degrees. This could be the result of dealumination during the synthesis of the zeolite paste due to the addition of the nitrate precursor, which are acidic salt. The mappings for all samples suggested the distribution of the metal dopants in the monoliths are even and no obvious aggregations of metal oxides were observed in the doped monoliths.

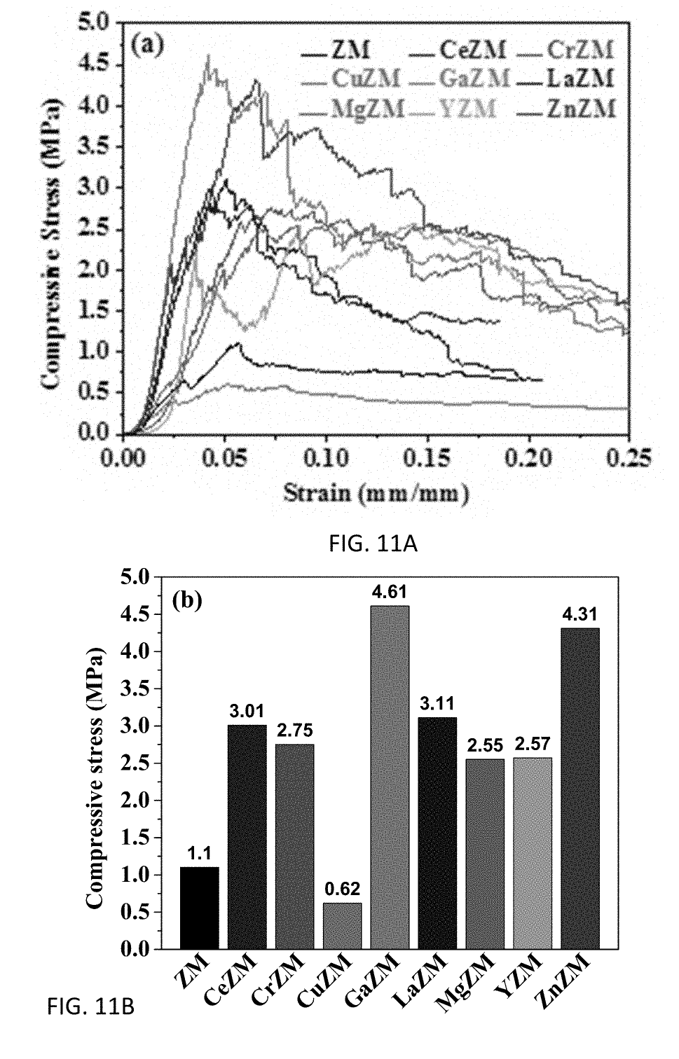

For the application in catalysis, the mechanical strength of the 3D-printed monolith is an important factor to consider. Compression testing results are depicted in FIG. 11A and FIG. 11B. As evident from FIG. 11A, the stress raised initially with increasing strain. It is illustrated in FIG. 11A that all M/ZSM-5 collapsed no later than the bare ZSM-5 monolith. The maximum stress after which a drop occurred was regarded as the critical stress that caused the monolith collapse. FIG. 11B compares the magnitude of compressive stress for various monolith and it can be concluded that various monoliths except for Cu/ZSM-5, all the doped monolith tolerated higher stress than the bare ZSM-5 before fracture.

Catalytic Test:

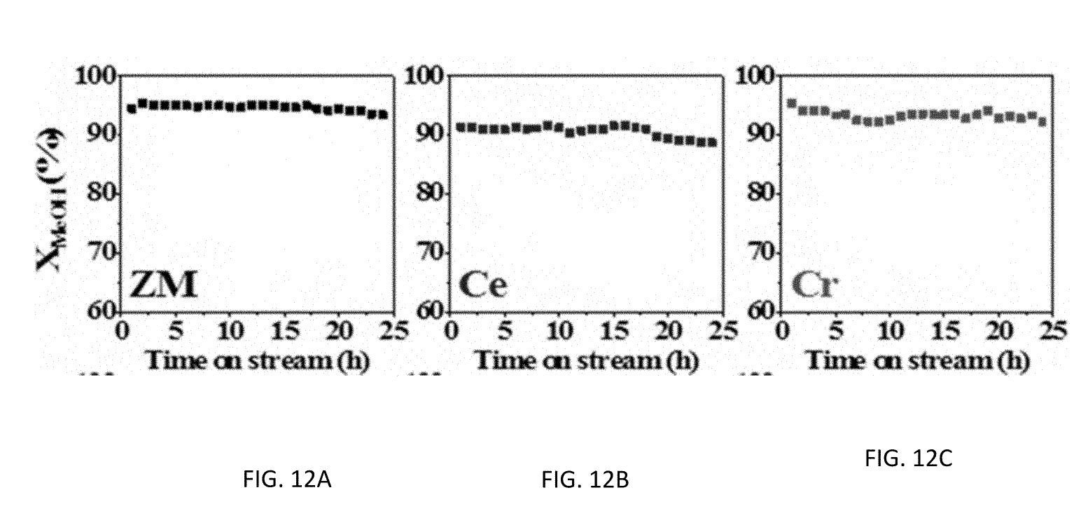

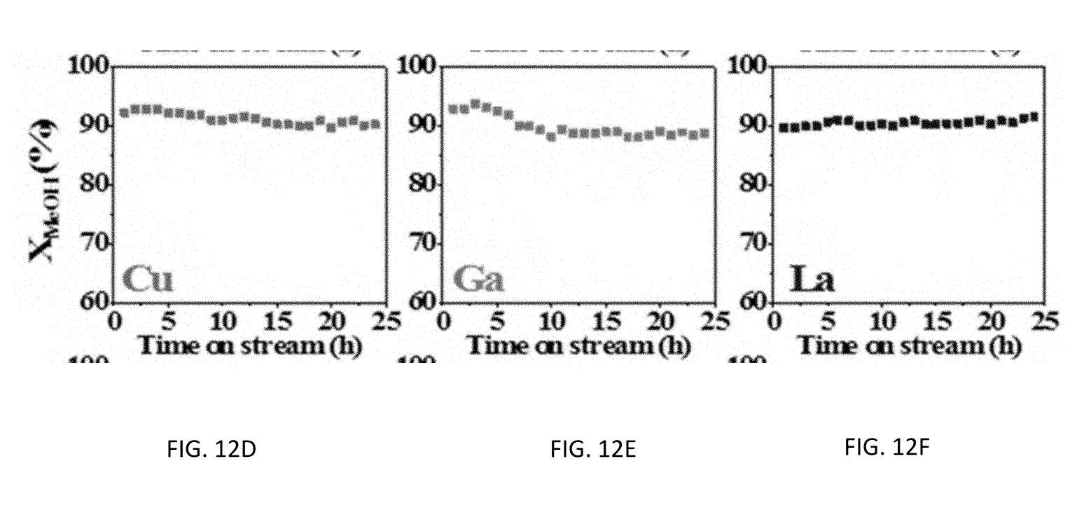

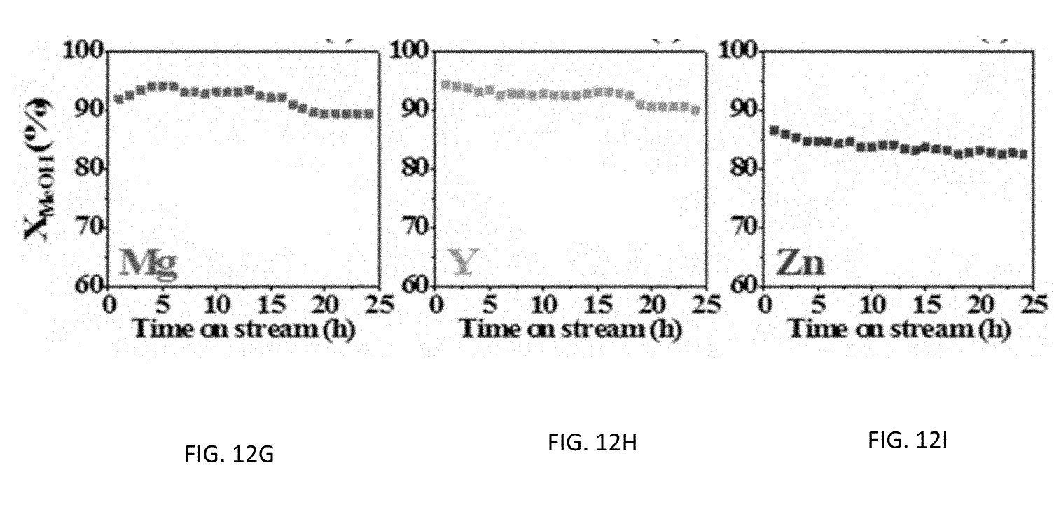

The performance of the zeolite monoliths as the catalyst for MTO process was evaluated at 673 K. The methanol conversion rates (X_{MeOH}) as a function of time on stream are displayed in FIG. 12. The ZSM-5 monolith showed the highest conversion of about 95% under the investigated conditions and the activity was stable within 24 hours. Cr/ZSM-5 exhibited comparable activity and stability with ZSM-5 while Ga/ZSM-5 resulted in a relatively high conversion of 93% at the initial stage of the reaction but deactivated rapidly with the conversion rate below 90%. Similar trend was found for Y/ZSM-5, with the conversion rate starting at 95% and reaching below 90%. Ce/, Cu/, La/, and Mg/ZSM-5 showed slightly lower activity and considerable stability within 24 h, with a constant conversion rate of 90%. Zn/ZSM-5 catalyzed the transformation of methanol with an initial conversion rate of ˜87% which later dropped to 82%. Considering the length of reaction time, all catalysts exhibited a stable activity in MTO process under 673 K.

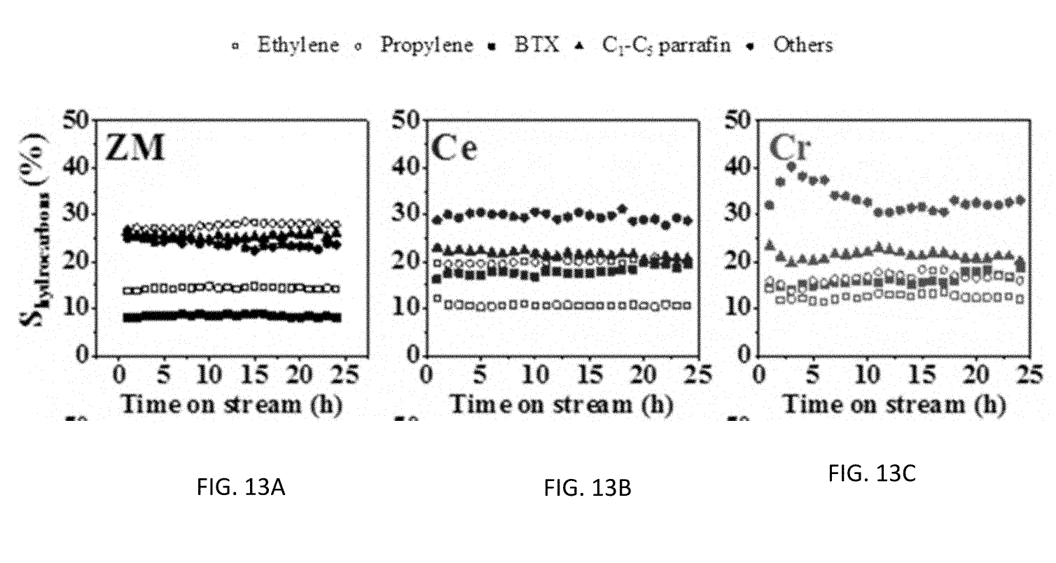

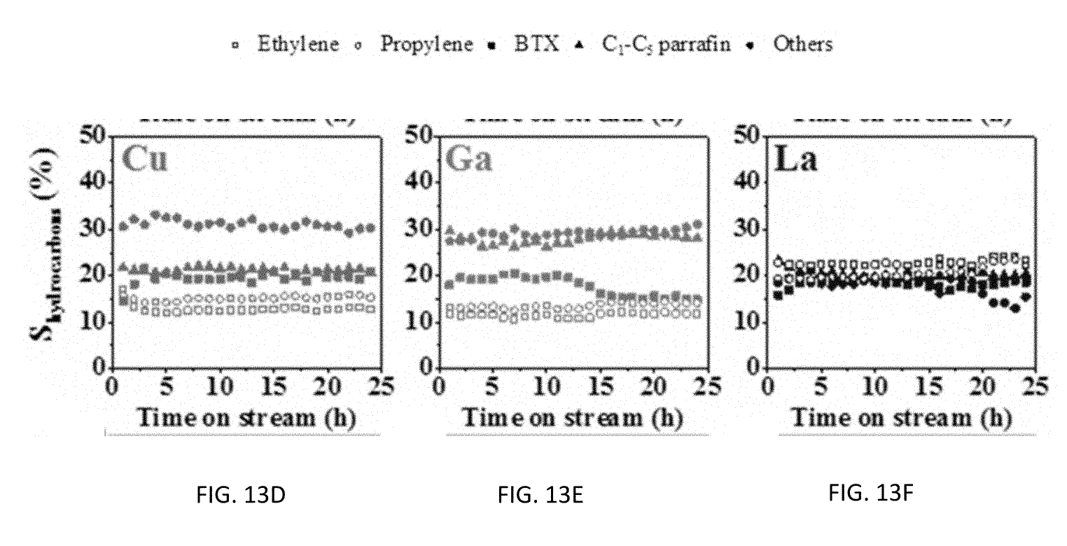

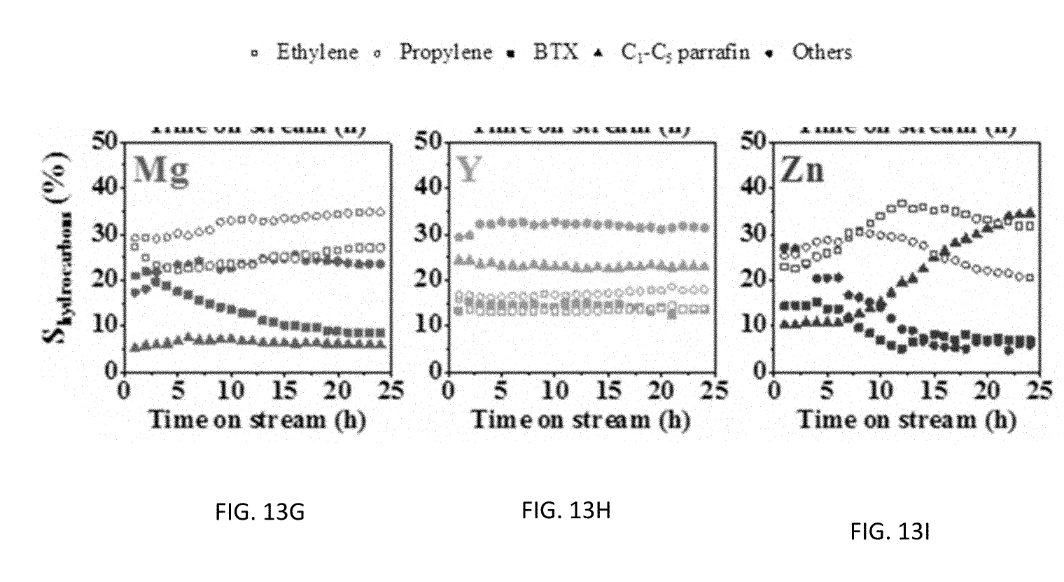

FIG. 13 shows the selectivity towards hydrocarbons in the MTO process. The product distribution trends are different on the investigated 3D-printed monoliths. Each metal uniquely affected the distribution. It can be clearly seen that, except for Mg/ and Zn/ZSM-5, all other metal-doped monoliths displayed decreased selectivity to light olefins compared to the bare ZSM-5. Selectivity to both ethylene and propylene were reduced over Ce/, Cr/, Cu, Ga/, and Y/ZSM-5. The reduction in light olefins occurred simultaneously with the increase in paraffin production, indicating the addition of these metal dopants benefited the secondary reaction to form paraffin, according to the mechanism of MTO process reported in previous research.⁶⁶ Unlike these dopants, selectivity to ethylene was increased over La/ZSM-5 while the selectivity to propylene was restrained. Notably, the enhancement of light olefins, both ethylene and propylene was significant over Mg/ZSM-5 and Zn/ZSM-5, while the production of the olefins was more stable and constant over Mg/ZSM-5. The enhanced selectivity to light olefins can be attributed to the altered acidity of the zeolite monoliths by the metal dopants. It is well known that strong Brønsted acid sites favor the conversion of methanol but also benefit the secondary reaction which eventually causes coke formation due to the oligomerization of the generated ethylene and propylene. It is widely agreed that moderate acidity favors the production of light olefins.¹ The Mg/ZSM-5 in this study, as has been demonstrated by the NH₃-TPD results, showed only weak acid sites, which could be the result of the interaction of Mg with the internal hydroxyl groups of the zeolites, thus, eliminating the strong Brønsted acid sites and forming Mg(OH)+ which contributes to the moderate acidity of the catalyst. Moreover, the Mg cation with relatively large radius and smaller atom weight makes it possible to occupy more space in micropores of the zeolite with the same weight percentage of loading, compared to other metal dopants if they also enter the micropores. This matches the results from the pore volume calculations. Previous work⁶⁷ highlighted that with more space taken by heteroatoms in the micropores, it is less likely to have coke deposition in the microporous zeolite because with these micropores, which act as the hydrocarbon pool, it is less likely to form aromatics which are the coke precursors.

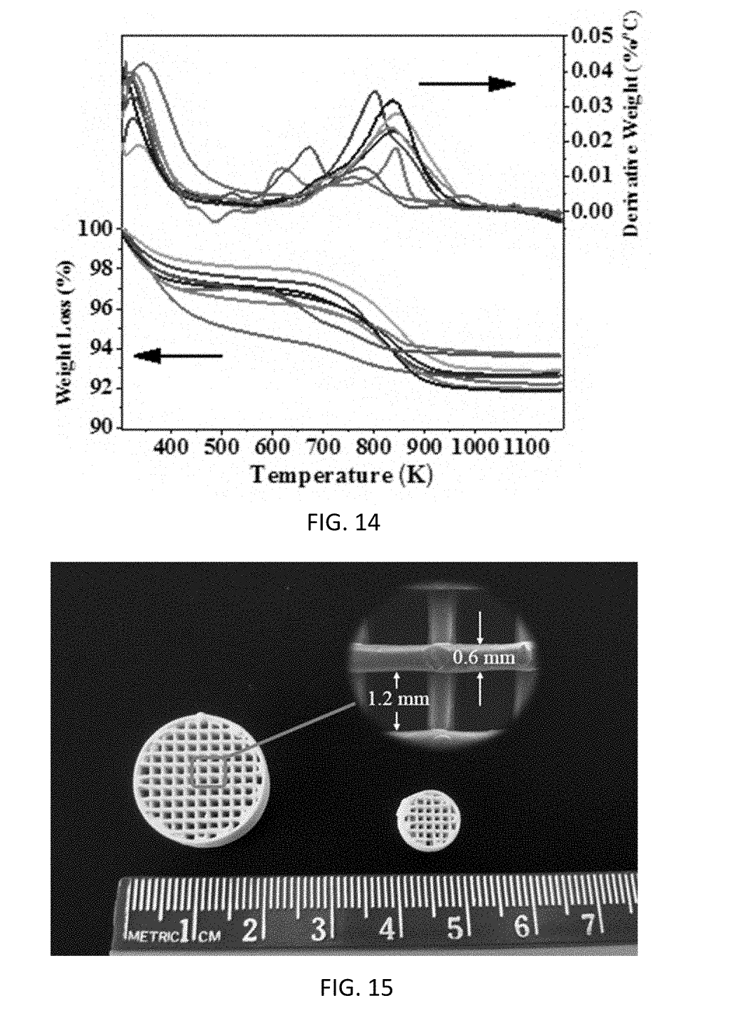

To verify the explanation with more evidence, TGA of the spent catalysts after 24 hours of methanol conversion at 673 K was carried out in the temperature range of 303 to 1173 K in a 60 mL min⁻¹ air flow. Both TGA and corresponding DTA profiles were plotted and displayed in FIG. 14. All samples experienced a weight loss close to 373 K, which was assigned to the moisture in the samples. The peaks for each sample in this region varied due to different adsorption ability for water in each spent catalyst. Mg/ZSM-5 exhibited two peak at around 753 K and 973 K with the intensity of the peaks much lower than most of the peaks observed for other monoliths. This suggests that the amount of coke deposition on Mg/ZSM-5 was the least among all the as-prepared M/ZSM-5 monoliths. In particular, the main peak at 753 K was lower than the main peak of Ce/, Cu/, Ga/, La/, Zn/ZSM-5 implying that the carbon number of the coke compounds was smaller than for other samples. It is noteworthy that the Cu/ZSM-5 experienced a weight increase under air flow at about 473 K which could be ascribed to the oxidation of CuO to Cu₂O₃ under such conditions.

### Conclusion

The 3D-printed monoliths with various dopants were prepared with a facile and rapid method. All samples retained their MFI framework after doping with metals. Most of the metals including Ga, La, Mg, Y, and Zn were well-dispersed in the zeolite without any measurable oxide crystal found by XRD. The as-prepared M/ZSM-5 monoliths exhibited macro-meso-micorporous network. Our results indicated that among all the 3D-printed monolith samples, Mg/ZSM-5 was the most affected sample, which showed moderate acid sites, occuping space in the micropores with relatively high mesopore volume. All these factors contributed to the high selectivity toward light olefins in the MTO process. The result of this investigation has proven that the Mg/ZSM-5 is a promising 3D-printed ZSM-5 monolith with metal incorporation for MTO process.

### REFERENCES

1. P. Tian, Y. Wei, M. Ye and Z. Liu, ACS Catalysis, 2015, 5, 1922-1938.
2. Z. Li, J. Martinez-Triguero, J. Yu and A. Corma, Journal of Catalysis, 2015, 329, 379-388.
3. X. Sun, S. Mueller, H. Shi, G. L. Haller, M. Sanchez-Sanchez, A. C. van Veen and J. A. Lercher, Journal of Catalysis, 2014, 314, 21-31.
4. R. Meiers, U. Dingerdissen and W. F. Hölderich, Journal of Catalysis, 1998, 176, 376-386.
5. N. M. Laurendeau, Progress in Energy and Combustion Science, 1978, 4, 221-270.
6. D. Sutton, B. Kelleher and J. R. H. Ross, Fuel Process. Technol., 2001, 73, 155-173.
7. M. Asadullah, S.-i. Ito, K. Kunimori, M. Yamada and K. Tomishige, Journal of Catalysis, 2002, 208, 255-259.
8. L. Wu, V. Degirmenci, P. C. M. M. Magusin, N. J. H. G. M. Lousberg and E. J. M. Hensen, Journal of Catalysis, 2013, 298, 27-40.
9. X. Li, A. Kant, Y. He, H. V. Thakkar, M. A. Atanga, F. Rezaei, D. K. Ludlow and A. A. Rownaghi, Catalysis Today, 2016, 276, 62-77.
10. C. Wang, Y. Chu, A. Zheng, J. Xu, Q. Wang, P. Gao, G. Qi, Y. Gong and F. Deng, Chemistry—A European Journal, 2014, 20, 12432-12443.
11. B. V. Vora, T. L. Marker, P. T. Barger, H. R. Nilsen, S. Kvisle and T. Fuglerud, in Studies in Surface Science and Catalysis, eds. M. de Pontes, R. L. Espinoza, C. P. Nicolaides, J. H. Scholtz and M. S. Scurrell, Elsevier, 1997, vol. 107, pp. 87-98.
12. G. Qi, Z. Xie, W. Yang, S. Zhong, H. Liu, C. Zhang and Q. Chen, Fuel Process. Technol., 2007, 88, 437-441.
13. D. Chen, K. Moljord, T. Fuglerud and A. Holmen, Microporous and Mesoporous Materials, 1999, 29, 191-203.
14. S. Ivanova, E. Vanhaecke, B. Louis, S. Libs, M.-J. Ledoux, S. Rigolet, C. Marichal, C. Pham, F. Luck and C. Pham-Huu, ChemSusChem, 2008, 1, 851-857.
15. A. A. Rownaghi and J. Hedlund, Industrial & Engineering Chemistry Research, 2011, 50, 11872-11878.
16. A. A. Rownaghi, F. Rezaei and J. Hedlund, Catalysis Communications, 2011, 14, 37-41.
17. A. A. Rownaghi, F. Rezaei, M. Stante and J. Hedlund, Applied Catalysis B: Environmental, 2012, 119-120, 56-61.
18. M. Bjørgen, F. Joensen, M. Spangsberg Holm, U. Olsbye, K.-P. Lillerud and S. Svelle, Applied Catalysis A: General, 2008, 345, 43-50.
19. P. L. Benito, A. G. Gayubo, A. T. Aguayo, M. Olazar and J. Bilbao, Journal of Chemical Technology & Biotechnology, 1996, 66, 183-191.
20. A. G. Gayubo, P. L. Benito, A. T. Aguayo, M. Olazar and J. Bilbao, Journal of Chemical Technology & Biotechnology, 1996, 65, 186-192.
21. M. Firoozi, M. Baghalha and M. Asadi, Catalysis Communications, 2009, 10, 1582-1585.
22. H.-G. Jang, H.-K. Min, J. K. Lee, S. B. Hong and G. Seo, Applied Catalysis A: General, 2012, 437-438, 120-130.
23. A. A. Rownaghi, F. Rezaei and J. Hedlund, Microporous and Mesoporous Materials, 2012, 151, 26-33.
24. S. Ivanova, B. Louis, B. Madani, J. P. Tessonnier, M. J. Ledoux and C. Pham-Huu, The Journal of Physical Chemistry C, 2007, 111, 4368-4374.
25. U. Olsbye, S. Svelle, M. Bjørgen, P. Beato, T. V. W. Janssens, F. Joensen, S. Bordiga and K. P. Lillerud, Angewandte Chemie International Edition, 2012, 51, 5810-5831.
26. B. Liu, L. France, C. Wu, Z. Jiang, V. L. Kuznetsov, H. A. Al-Megren, M. Al-Kinany, S. A. Aldrees, T. Xiao and P. P. Edwards, Chemical Science, 2015, 6, 5152-5163.
27. M. Kaarsholm, F. Joensen, J. Nerlov, R. Cenni, J. Chaouki and G. S. Patience, Chemical Engineering Science, 2007, 62, 5527-5532.
28. V. R. Choudhary, K. C. Mondal and S. A. R. Mulla, Angewandte Chemie, 2005, 117, 4455-4459.
29. J.-C. Lin, K.-J. Chao and Y. Wang, Zeolites, 1991, 11, 376-379.
30. D. Goto, Y. Harada, Y. Furumoto, A. Takahashi, T. Fujitani, Y. Oumi, M. Sadakane and T. Sano, Applied Catalysis A: General, 2010, 383, 89-95.
31. B. Kaur, M. Tumma and R. Srivastava, Industrial & Engineering Chemistry Research, 2013, 52, 11479-11487.
32. K. Ramesh, C. Jie, Y.-F. Han and A. Borgna, Industrial & Engineering Chemistry Research, 2010, 49, 4080-4090.
33. G. Coudurier, A. Auroux, J. C. Vedrine, R. D. Farlee, L. Abrams and R. D. Shannon, Journal of Catalysis, 1987, 108, 1-14.
34. R. M. Barrer, J. W. Baynham, F. W. Bultitude and W. M. Meier, Journal of the Chemical Society (Resumed), 1959, DOI: 10.1039/J R9590000195, 195-208.
35. D. W. Breck and J. V. Smith, Scientific American, 1959, 200, 85-96.
36. G. Kinger, A. Lugstein, R. Swagera, M. Ebel, A. Jentys and H. Vinek, Microporous and Mesoporous Materials, 2000, 39, 307-317.
37. H. Thakkar, S. Eastman, A. Hajari, A. A. Rownaghi, J. C. Knox and F. Rezaei, ACS Applied Materials & Interfaces, 2016, 8, 27753-27761.
38. X. Li, W. Li, F. Rezaei and A. Rownaghi, Chemical Engineering Journal, 2018, 333, 545-553.
39. S. Couck, J. Cousin-Saint-Remi, S. Van der Perre, G. V. Baron, C. Minas, P. Ruch and J. F. M. Denayer, Microporous and Mesoporous Materials, 2018, 255, 185-191.
40. H. Thakkar, S. Eastman, A. Al-Mamoori, A. Hajari, A. A. Rownaghi and F. Rezaei, ACS Applied Materials & Interfaces, 2017, 9, 7489-7498.
41. H. Thakkar, S. Eastman, Q. Al-Naddaf, A. A. Rownaghi and F. Rezaei, ACS Applied Materials & Interfaces, 2017, 9, 35908-35916.
42. C. R. Tubío, J. Azuaje, L. Escalante, A. Coelho, F. Guitián, E. Sotelo and A. Gil, Journal of Catalysis, 2016, 334, 110-115.
43. N. Hadi, A. Niaei, S. R. Nabavi, M. Navaei Shirazi and R. Alizadeh, Journal of Industrial and Engineering Chemistry, 2015, 29, 52-62.
44. M. Conte, J. A. Lopez-Sanchez, Q. He, D. J. Morgan, Y. Ryabenkova, J. K. Bartley, A. F. Carley, S. H. Taylor, C. J. Kiely, K. Khalid and G. J. Hutchings, Catalysis Science & Technology, 2012, 2, 105-112.
45. M. Li, Y. Zhou, I. N. Oduro and Y. Fang, Fuel, 2016, 168, 68-75.
46. I. A. Bakare, O. Muraza, M. Yoshioka, Z. H. Yamani and T. Yokoi, Catalysis Science & Technology, 2016, 6, 7852-7859.
47. C. Xu, B. Jiang, Z. Liao, J. Wang, Z. Huang and Y. Yang, RSC Advances, 2017, 7, 10729-10736.
48. J. Lee, U. G. Hong, S. Hwang, M. H. Youn and I. K. Song, Fuel Process. Technol., 2013, 109, 189-195.
49. B. Liu, D. Slocombe, M. AlKinany, H. AlMegren, J. Wang, J. Arden, A. Vai, S. Gonzalez-Cortes, T. Xiao, V. Kuznetsov and P. P. Edwards, Applied Petrochemical Research, 2016, 6, 209-215.
50. A. A. Lappas, C. S. Triantafillidis, Z. A. Tsagrasouli, V. A. Tsiatouras, I. A. Vasalos and N. P. Evmiridis, in Studies in Surface Science and Catalysis, eds. R. Aiello, G. Giordano and F. Testa, Elsevier, 2002, vol. 142, pp. 807-814.
51. M. Liu, J. Li, W. Jia, M. Qin, Y. Wang, K. Tong, H. Chen and Z. Zhu, RSC Advances, 2015, 5, 9237-9240.
52. S. Lai, D. Meng, W. Zhan, Y. Guo, Y. Guo, Z. Zhang and G. Lu, RSC Advances, 2015, 5, 90235-90244.
53. F. Rahmani and M. Haghighi, RSC Advances, 2016, 6, 89551-89563.
54. S. Meghana, P. Kabra, S. Chakraborty and N. Padmavathy, RSC Advances, 2015, 5, 12293-12299.
55. N. Mimura, I. Takahara, M. Inaba, M. Okamoto and K. Murata, Catalysis Communications, 2002, 3, 257-262.
56. F. A. Hasan, P. Xiao, R. K. Singh and P. A. Webley, Chemical Engineering Journal, 2013, 223, 48-58.
57. D. Liu, W. Zhou and J. Wu, Chemical Engineering Journal, 2016, 284, 862-871.
58. M. A. Ali, B. Brisdon and W. J. Thomas, Applied Catalysis A: General, 2003, 252, 149-162.
59. J. C. Jansen, F. J. van der Gaag and H. van Bekkum, Zeolites, 1984, 4, 369-372.
60. S. Allahyari, M. Haghighi, A. Ebadi and S. Hosseinzadeh, Ultrasonics Sonochemistry, 2014, 21, 663-673.
61. I. C. L. Barros, V. S. Braga, D. S. Pinto, J. L. de Macedo, G. N. R. Filho, J. A. Dias and S. C. L. Dias, Microporous and Mesoporous Materials, 2008, 109, 485-493.
62. Z. Liu, Z. Zhang, W. Xing, S. Komarneni, Z. Yan, X. Gao and X. Zhou, Nanoscale Research Letters, 2014, 9, 550.
63. H. Chen, J. Wydra, X. Zhang, P.-S. Lee, Z. Wang, W. Fan and M. Tsapatsis, Journal of the American Chemical Society, 2011, 133, 12390-12393.
64. M. Impéror-Clerc, P. Davidson and A. Davidson, Journal of the American Chemical Society, 2000, 122, 11925-11933.
65. R. W. Borry, Y. H. Kim, A. Huffsmith, J. A. Reimer and E. Iglesia, The Journal of Physical Chemistry B, 1999, 103, 5787-5796.
66. E. Vera-Castaneda, Study of the methanol conversion to ethylene and propylene using small pore size zeolites, Texas A and M Univ., College Station (USA), 1985.
67. C. H. Collett and J. McGregor, Catalysis Science & Technology, 2016, 6, 363-378.

### Example 2: 3D-Printed Zeolite Monoliths with Macro-Meso-Microporosity for Selective Methanol to Light Olefins Reaction

### Introduction

Methanol-to-olefins (MTO) conversion is an important reaction to produce light olefins such as ethylene and propylene. Zeolites are generally the most widely used catalyst for this reaction mainly due to their tunable acidity, unique porosity and designable configuration [1-4]. Among various zeolite, SAPO-34 silico-aluminophosphate (CHA structure) has been proven to be an efficient catalyst that exhibits high selectivity towards light olefins as a result of its proper acid site strength and three-dimensional cage structure with 3.8 Å×3.8 Å eight-ring channels [5]. However, severe coke formation over this catalyst is usually observed which limits its widespread use [6]. To extend the catalyst lifetime, HZSM-5 zeolite (MFI structure) with larger channels (4.7 Å×4.5 Å) has been suggested as an alternative due to its relatively high olefins selectivity [7]. Endeavors have been made to increase olefin yield over HZSM-5 zeolite through two approaches: modification of the catalytic components and change of the catalyst configuration. The realization of the former is usually through introduction of heteroatoms and optimization of SiO₂/Al₂O₃ ratio, while the latter is through alteration of particle size and porosity, and fabrication of structured catalyst [8-14].

As a major type of structured catalysts, monolith catalysts, a block of structured material which contains various types of interconnected or separated channels, have been mainly applied for environmental applications [15] such as removal of SOₓ/NOₓ from automotive exhaust gases [16-19] and selective catalytic reduction (SCR) process [20-23]. With low pressure drop, high thermal stability, great mechanical integrity, and good mass transfer characteristics, monolithic catalysts are promising alternative to conventional pellets and beads. More importantly, the diffusion efficiency of catalysts can be remarkably improved [24]. Such structures have been previously utilized in MTO reaction. For instance, Li et al. [25] synthesized ZSM-5 monolith with tetramodel porosity and the catalyst showed good activity and selectivity to propylene. In another study, Ivanova et al. [12] coated ZSM-5 zeolite on β-Sic monolith and tested the supported catalyst in the conversion of methanol into light olefins. It was found that structured zeolite packings exhibited higher activity and selectivity than the powdered zeolite prepared under the same synthesis conditions. The comparison between the ZSM-5 monolith foam and its pelletized form by Lee et al. [26] illustrated that structured catalyst displayed higher selectivity to light olefins with enhanced mass transport characteristics.

Monolithic catalysts are conventionally prepared via extrusion process [27-30]. Unique dies with specific sizes and shapes are dispensable for this approach and thus restrict the diversity of catalyst configuration and enhance total fabrication costs. With the emerging three-dimensional (3D) printing technique and its broad application in fabricating various materials [31-35], the preparation of monolithic catalysts via this method opens new opportunities. Precise fabrication with desired configuration, high productivity and low fabrication cost are the advantages of this technique. Recently, Tubio et al. used 3D-printing technique for preparation of a heterogeneous copper-based catalyst [31]. The structured catalyst showed high mechanical strength, requirement-meeting reactivity and possible recyclability in a model Ullmann reaction. Lefevere et al. prepared an stainless steel support using three dimensional fibre deposition (3DFD) technology and washcoated it with zeolite. The coated structured catalysts showed beneficial effect on the selectivity and activity of the catalyst in the conversion of methanol to light olefins [32]. Rezaei and coworkers successfully prepared monoliths of porous materials like zeolites, aminosilicates and metal-organic frameworks (MOFs) and utilized them for CO₂ adsorption. The monoliths displayed excellent adsorption uptake comparable to that of powder sorbents [33-35]. Considering its significance in catalytic reactions, the study of 3D-printed monolithic zeolite catalyst is scarce.

Motivated by the facility of 3D printing technique to prepare monolith catalysts, we synthesized HZSM-5 monolith using our lab-scale 3D printer. To improve the catalyst performance, SAPO-34 crystals was grown on the monoliths using secondary growth approach. The characterizations of the 3D-printed monoliths were carried out by various techniques such as XRD, SEM, N₂ physisorption, NH₃-TPD, and compressive test. The catalytic performance of the 3D-printed monoliths were tested in MTO reaction.

### Experimental

Preparation of 3D-Printed Zeolite Monoliths:

Monoliths of HZSM-5 zeolite (M1) and HZSM-5 diluted with amorphous silica (M2) were synthesized from commercial ammonia-ZSM-5 powder (CBV 5524G, Zeolyst, SiO₂/Al₂O₃=50) and amorphous silica (Tixosil). Ammonia-ZSM-5 was calcined at 823 K for 6 hours to produce parent HZSM-5 powder. The desired amounts of HZSM-5/silica powder were stirred with bentonite clay (Sigma-Aldrich), which was used as a binder [36], using a high-performance agitator (Model IKA-R25). Sufficient distilled water was then added until a homogeneous slurry was obtained. The aqueous paste with extrudable viscosity was obtained after adding methyl cellulose (Thermo Fisher), as a plasticizer, with sufficient stir. The paste was loaded into a 10 mL syringe (Techcon Systems) furnished with a nozzle of 0.60 mm in diameter. The synthesis of the monolith was carried out on a lab-scale 3D printer, prior to which the program of printing paths was designed by AutoCAD software and coded by Slic3r. The paste was dispensed and deposited on an alumina substrate layer-by-layer to form a honeycomb-like monolith. The fresh 3D-printed monoliths were dried overnight and then calcined for 6 hours at 873 K to remove methyl cellulose. The uniform cylindrical monolith possessed 50% infill density leading to a 0.60 mm wall thickness and 1.20 mm² channel length. The optical image of the monolith is shown in FIG. 15 and the composition of the samples are listed in Table 2. Both M1 and M2 were prepared in two sizes. Samples with 20 mm in diameter were used for characterization while 10 mm sizes were used for catalyst tests. It is worth mentioning here that to study the effect of amorphous silica on the monoliths properties and performance, a silica monolith was also prepared, denoted as M3, for mechanical strength investigation, although it is barely active. Parent H form of ZSM-5 power is denoted as ZP form comparison with the monolith samples in certain cases.

TABLE 2
Composition of the 3D-printed monoliths.

| | | | | Bentonite | Methyl | |
|---|---|---|---|---|---|---|
| | | HZSM-5 | silica | Clay | Cellulose | Loading |
| NO. | Sample | (wt. %) | (wt. %) | (wt. %) | (wt. %) | (wt. %) |
| 1 | M1 | 87.50 | — | 10 | 2.5 | — |
| 2 | M2 | 43.75 | 43.75 | 10 | 2.5 | — |
| 3 | M3 | — | 87.50 | 10 | 2.5 | — |
| 4 | SPM1 | 87.50 | — | 10 | 2.5 | 4.6% |
| 5 | SPM2 | 43.75 | 43.75 | 10 | 2.5 | 5.8% |

Growth of SAPO-34 on 3D-Printed Zeolite Monoliths:

PANalytical X'Pert Multipurpose X-ray Diffractometer was used to obtain the X-ray diffraction (XRD) patterns. It was operated at 40 kV and 40 mA with Cu-Kα1 monochromatized radiation (λ=0.154178 nm) and the scanned angle range (2θ) from 5° to 50° at a rate of 2.0° min⁻¹. Textural properties such as total surface area, external surface area and pore size distributioin (PSD) were measured using Brunauer-Emmett-Teller (BET) equation, t-plot and Barrett-Joyner-Halenda (BJH) methods, respectively, based on the N₂ physisorption analysis carried out by a Micromeritics 3Flex surface characterization analyzer at 77 K. Before the measurements, all samples were degassed at 573 K for 6 hours. Morphology of the materials was analyzed with a field-emission scanning electron microscopy (Hitachi S-4700). Temperature-programmed desorption of ammonia (NH₃-TPD) was carried out to evaluate acid properties. NH₃ adsorption was performed under a flow of 5 vol % NH₃/He. The desorption of NH₃ was measured from 373 K to 873 K at a heating rate of 10 K min⁻¹. A mass spectroscopy (MicrotracBEL, BELMass) was used to detect the quantity of NH₃ desorption. The Brønsted and Lewis acid sites were estimated by ex-situ pyridine-adsorption Fourier-transform infrared spectroscopy (FTIR) using a Bruker Tensor spectrophotometer. The catalysts were firstly activated at 673 K for 4 hours to remove moisture and then cooled down to 313 K for adsorption of pyridine until saturation. Since all monolith samples contained bentonite clay (with high proportion of Al), as a binder, it was not possible to obtain reasonable results with ²⁷Al MAS NMR and therefore this measurement was not included. However, since Si and Al are highly correlated in the zeolite framework, ²⁹Si MAS NMR, can reflect the stability of the samples. Magic angle spinning (MAS) nuclear magnetic resonance (NMR) spectra of ²⁹Si (²⁹Si MAS NMR) were obtained using a Bruker 400 MHz FT spectrometer. The spectra were collected using a 4 mm probe spinning at 10 kHz. Mechanical testing was performed with an Instron 3369 (Instron, Norwood, Mass., USA) mechanical testing device. Monolith samples were polished with smoothing sandpaper to provide smooth and parallel surfaces. Then they were placed between two metal plates and compressed with a 500 N load cell at 2.5 mm/min while the applied load and displacement of the monolith surfaces were recorded. The spent catalysts after MTO reaction were analyzed by thermogravimetric analysis-differential thermal analysis (TGA-DTA) using a Q500, TA Instruments. The temperature was raised from 303 K to 1173 K, at a rate of 10 K/min in a 60 mL min⁻¹ air flow.

Catalytic Test:

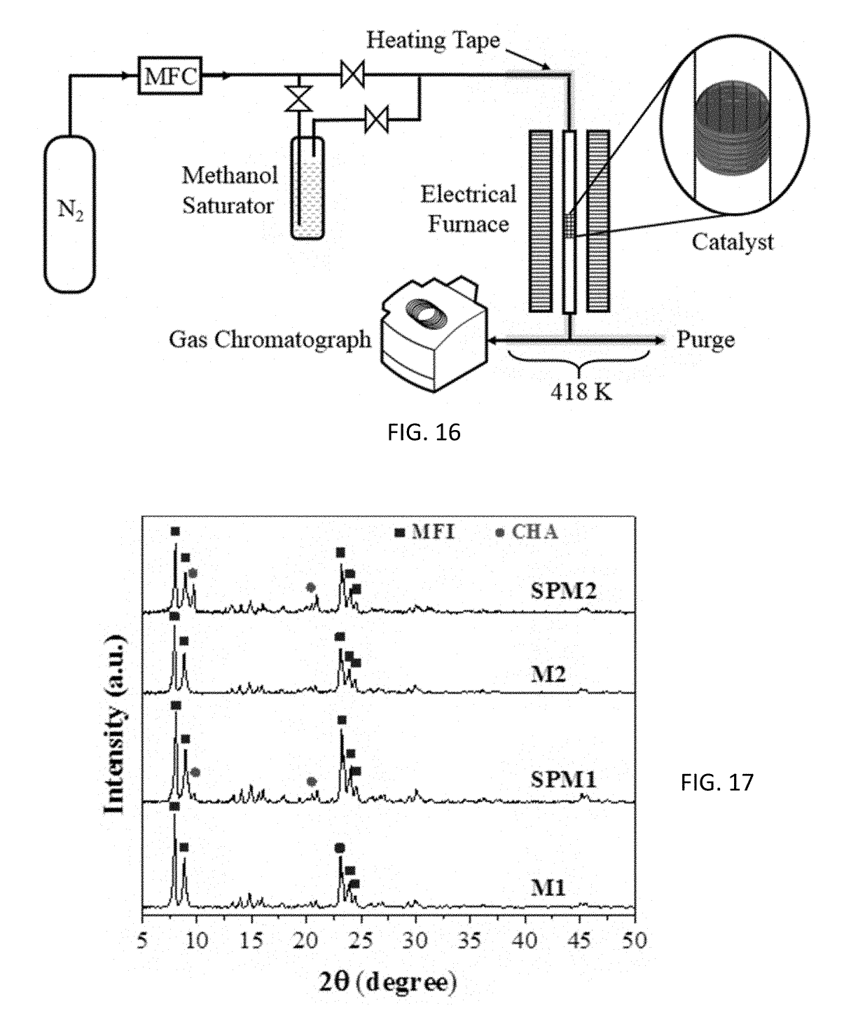

The 3D-printed zeolite monoliths and their powder counterparts were tested in MTO reaction. The setup of the fixed-bed reactor is shown in FIG. 16. A flow of nitrogen, acted as carrier gas, saturated with methanol at 303 K was fed to a stainless steel fixed-bed reactor. The flow rate was controlled by a mass flow controller (Brooks, 5850). About 0.3 g of each catalyst was tested under 623 K and 673 K at 1.01 bar with different weight hourly space velocity (WHSV) of 0.35 h⁻¹ and 1.06 h⁻¹. The reactor is 10 mm in diameter and 300 mm in length. The catalysts were firstly activated in-situ at 823 K in nitrogen flow for 2 hours. The products were analyzed every hour using an on-line gas chromatography (SRI 8610C) equipped with a flame ionized detector (GC-FID) connected to mxt-wax/mxt-alumina capillary column for hydrocarbons. The inlet tube was kept heated at 383 K using a heating tape. Moreover, the effluent line of the reactor till GC injector was kept at 418 K to avoid potential condensation of hydrocarbons.

### Results and Discussion

Characterization of 3D-Printed Zeolite Monoliths:

The XRD patterns of all the as-synthesized monoliths are shown in FIG. 17. As illustrated, the crystallinity of the zeolite was well retained after formulation into the monoliths. For M1 and M2 samples, characteristic peaks of MFI structure at 2θ=7.96°, 8.88°, 23.2°, and 23.9° were identified which correspond to (101), (200), (501), and (303) planes respectively. The weaker intensity of the peaks in M2 spectrum than in M1 stems from the dilution of zeolite with silica [38]. Additional CHA characteristic peaks at 28=9.4° and 20.5° in the spectra of SPM1 and SPM2 samples suggest the existence of SAPO-34 crystals. Since these peaks refer to (101) and (211) planes respectively, the predominant peak at 28=9.4° indicates that the growth of the SAPO-34 crystals was highly oriented on all the monoliths and the results are in accordance with previously reported works [39, 40].

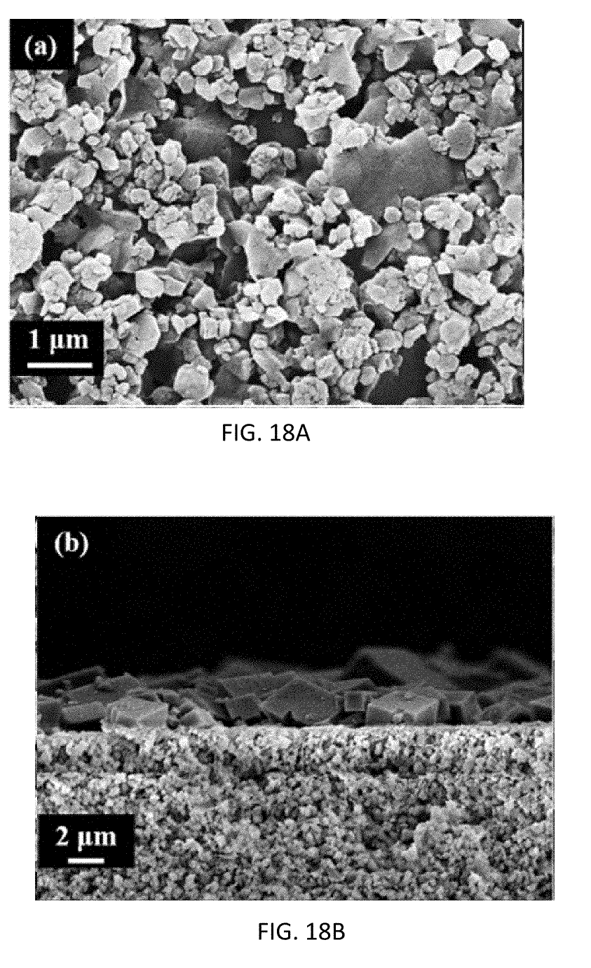

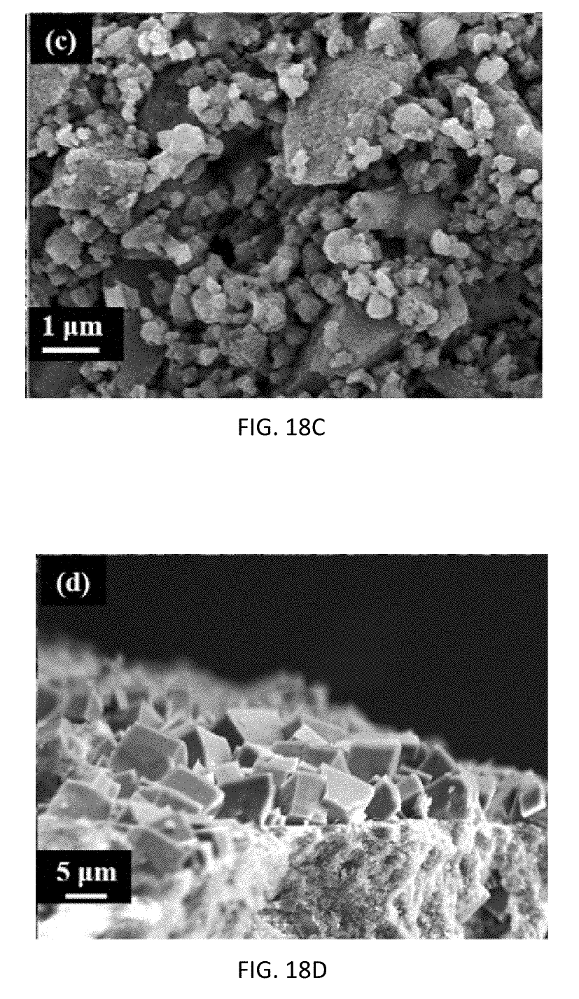

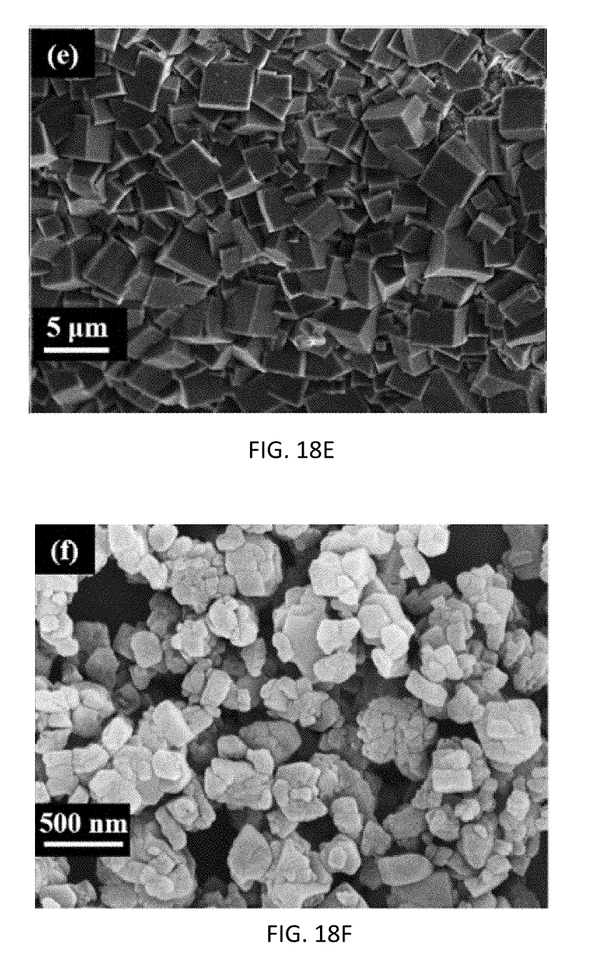

FIG. 18A shows the morphology of the 3D-printed HZSM-5 monolith (M1). Compared with the parent HZSM-5 powder in FIG. 18F, bulky agglomeration of HZSM-5 particles was observed in M1 due to the use of binder and the calcination of the monolith. The networks of sintered zeolite particles formed a porous structure with voids of sizes ranging from 200 nm to 1000 nm [33]. The removal of methyl cellulose after calcination also generated mesopores in monoliths. The M2 monolith in FIG. 18C shows similar morphology to the M1 sample but has rougher surface. The distinction in surface properties is more apparent in FIG. 18B and FIG. 18D under smaller magnifications in which the monoliths were used as the substrate for SAPO-34 growth. The rough surface of M2 might be attributed to the small size of silica particles (17.4 nm). FIG. 18B and FIG. 18E are respectively the cross sectional and top view of SPM1. It is clearly illustrated that compact cubic CHA particles were grown on the monolith surface, forming a SAPO-34 layer. The average particle size is 3.4 μm and the thickness of the SAPO-34 crystal layer is approximately 5.0 μm. Unlike those reported randomly orientated SAPO-34 membranes with even distributed XRD peaks [40-42], the SAPO-34 crystals in this work have regular cubic outline, which further verifies the formation of highly oriented crystals, in agreement with the XRD patterns. The cross sectional view of SPM2 shown in FIG. 18D confirms a film thickness of 10.0 μm for SAPO-34 layer. Previous investigations have shown that the support surface properties have significant effects on the thickness of grown crystals [43]. Herein, it is proposed that due to the employment of silica in SPM2, there are more mesopores formed on the monolith surface, which can be later seen in nitrogen physical sorption analysis, providing more voids for SAPO-34 seeds to grow. This is also reflected by the greater CHA peak intensity of the SPM2 at 2θ=9.4° than that of SPM1.

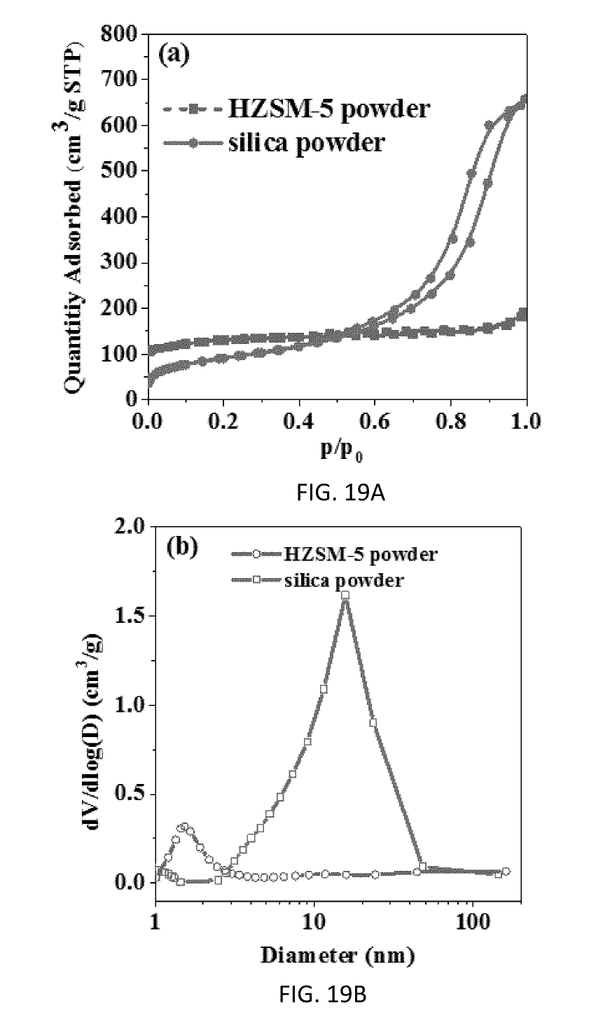

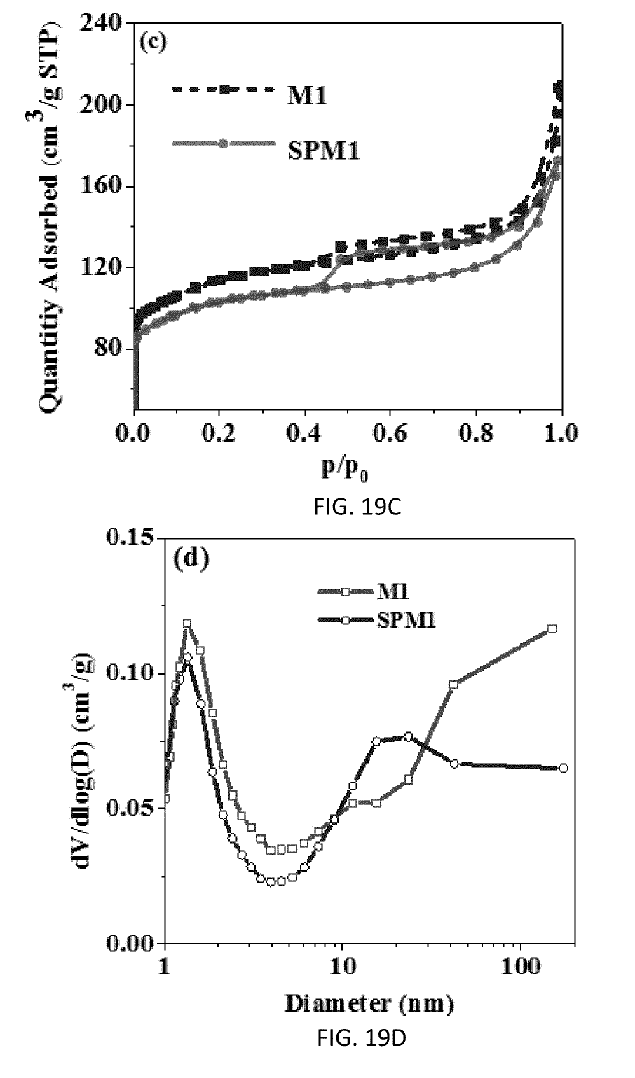

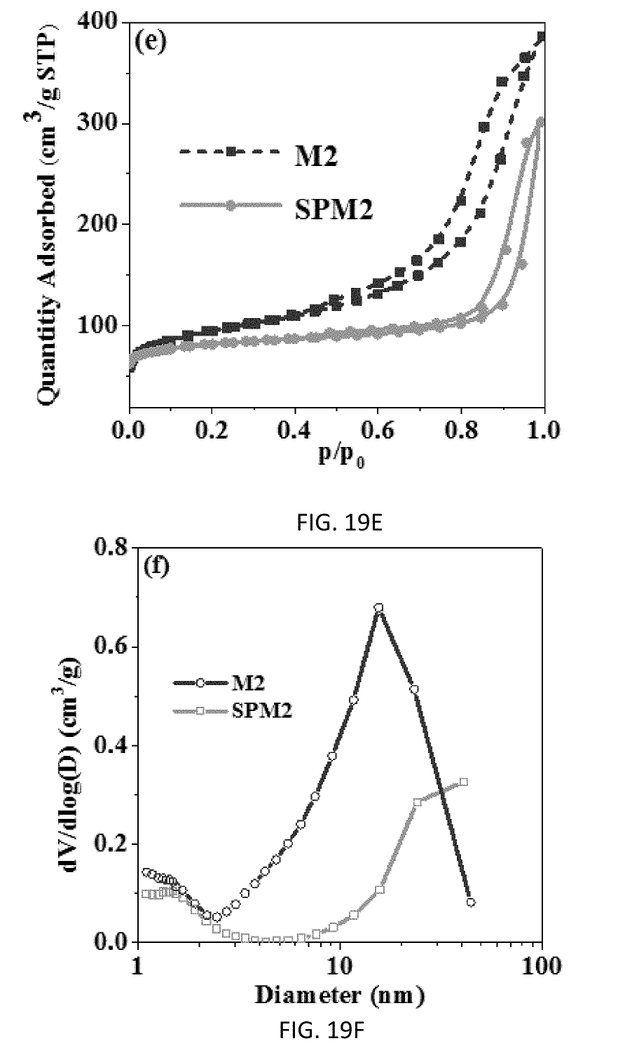

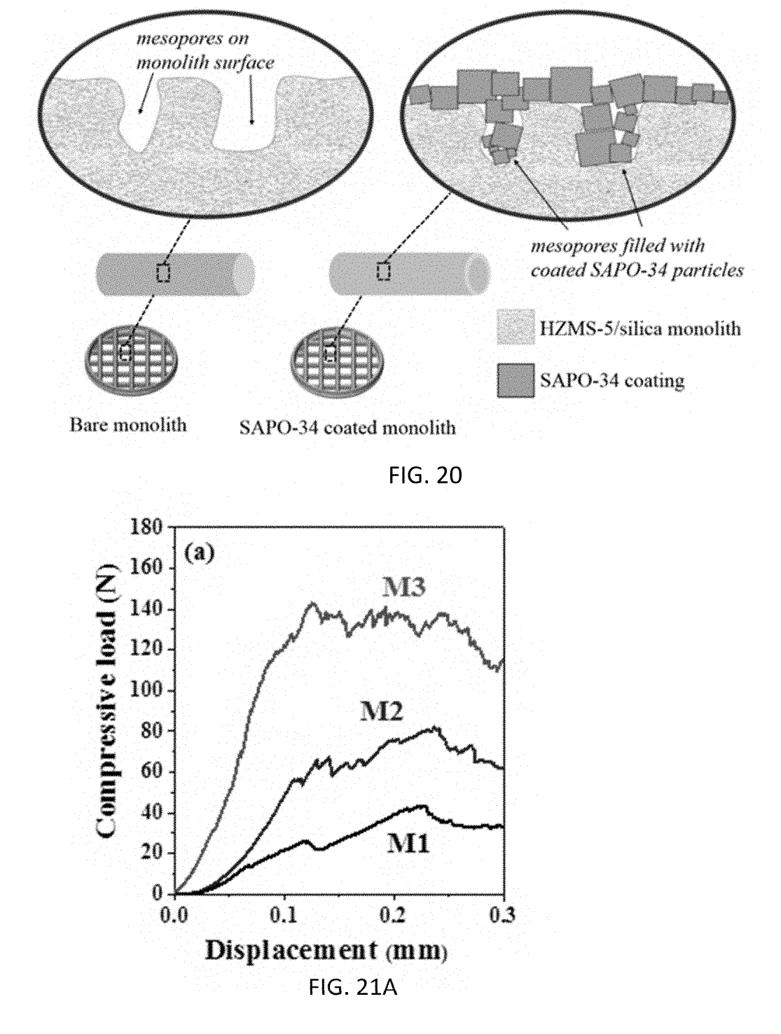

Table 3 shows the physical properties of all monoliths and their parents HZSM-5 and silica powders. Corresponding nitrogen physical sorption isotherms and PSD are displayed in FIG. 19A, FIG. 19B, FIG. 19C, FIG. 19D, FIG. 19E, and FIG. 19F. Silica was featured by its high surface area of 326 m²/g, of which 305 m²/g was contributed from external surface. Considering the nano-scale particle size (12±nm) provided by the supplier, the outstanding high mesopore volume was generated in monolith. The comparison between the particle size of HZSM-5 powder and M1 sample in FIG. 19B and FIG. 19D implies that the micropore volume decreased while mesopores volume increased after zeolite formulation into the monolith. This is mainly due to the addition of the binder and the removal of the methyl cellulose respectively. The M2 monolith displayed large mesopore volume of 0.54 cm³/g as a result of silica. Both M1 and M2 are demonstrated in FIG. 19A, FIG. 19B, FIG. 19C, FIG. 19D, FIG. 19E, and FIG. 19F to have hierarchical porosity [44, 45]. In addition to the 1.2 mm channels, the 3D printing technique produced monoliths with macro-meso-micropores [46, 47]. FIG. 19D together with pore volume values of M1 and SPM1 in Table 3 suggest that the addition of SAPO-34 layer decreased the porosity of the monoliths. Since the SAPO-34 crystals were grown from seeds, which were small enough to fill the mesopores, the CHA crystals might cause pore clogging when growing, as is shown in FIG. 20. This effect is more significant in M2 and SPM2 pair because more mesopores exist in M2 than the corresponding M1 monolith.

TABLE 3
N₂ physisorption data of the bare (M1, M2) and SAPO-
coated (SPM1, SPM2) 3D-printed monoliths.

| | S_{BET}^{a} | S_{mic}^{b} | Sₑₓₜₑᵣₙₐₗ | Vₜₒₜ^{c} | V_{mic}^{b} | Vₘₑₛₒ |
|---|---|---|---|---|---|---|
| Samples | (m²/g) | (m²/g) | (m²/g) | (cm³/g) | (cm³/g) | (cm³/g) |
| ZP | 429 | 261 | 168 | 0.30 | 0.13 | 0.17 |
| silica | 326 | 21 | 305 | 1.02 | 0.01 | 1.01 |
| M1 | 372 | 213 | 159 | 0.30 | 0.11 | 0.19 |
| SPM1 | 336 | 206 | 130 | 0.27 | 0.10 | 0.17 |
| M2 | 323 | 119 | 204 | 0.60 | 0.06 | 0.54 |
| SPM2 | 269 | 174 | 95 | 0.47 | 0.09 | 0.38 |

^{a}S_{BET} was obtained by analyzing nitrogen adsorption data at 77K in a relative vapor pressure ranging from 0.05 to 0.3.
^{b}Micropore area and micropore volume were determined using t-plot method.
^{c}Total pore volume was estimated based on the volume adsorbed at p/p₀ = 0.99.

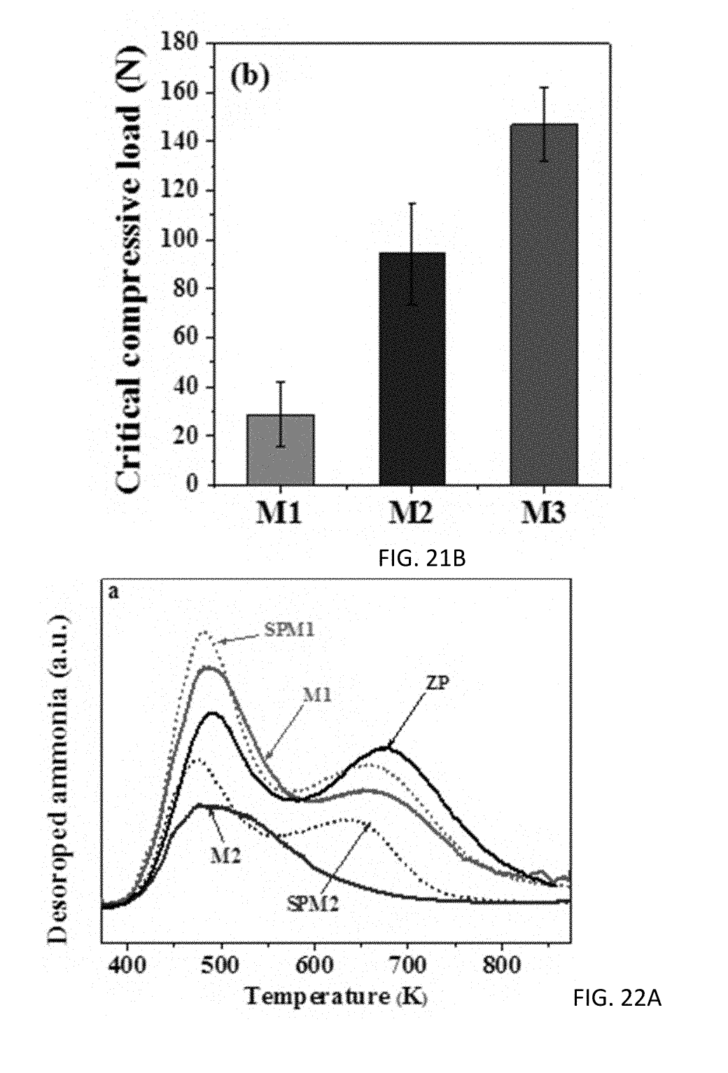

The mechanical strength of all the monolith catalysts was assessed by compression test. The sample size used in this test was 10 mm in diameter with 50% infill density. FIG. 21A shows the compressive load on the monoliths as a function of displacement of the monolith surfaces in axial direction. Initially, the displacement was increased with the applied load. The maximum force after which a drop occurred is regarded as the critical load that causes the monolith collapse. Three specimens were tested for each sample and the critical load for various samples are shown in FIG. 21B. It is obvious that with higher silica content the monoliths tend to be stronger. It is attributed to the small size of the parent silica powder which underwent less deformation when force was applied [48].

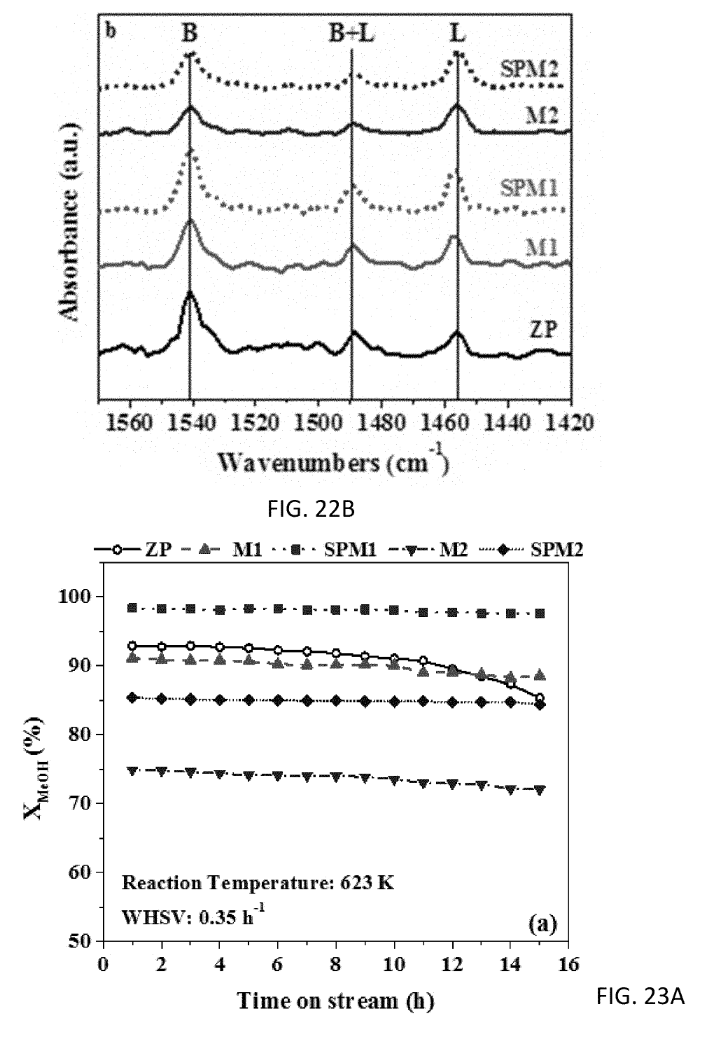

The NH₃-TPD profiles of the zeolite powder, the bare monoliths and SAPO-34 grown monoliths are presented in FIG. 22A. All samples show acid sites with two strengths: weak acid sites from which ammonia was desorbed at 474-490 K and strong acid sites at 629-676 K, as listed in Table 4. The comparison between ZP and M1 suggests that formulation into monolith decreased strong acid site and increased weak acid site simultaneously. M2 (0.220 mmol g⁻¹) possessed almost half of the acid sites amount as compared with M1 (0.365 mmol g⁻¹) due to the presence of silica in its structure. Both weak and strong acid sites were decreased due to the incorporation of the amorphous silica. The fact that strong acid sites of M2 were barely observed suggests that silica employment not only affects the acid site amount but also the acid strength. The growth of SAPO-34 on both monoliths enhanced the total amount of acid sites on the monolith surface. Notably, the amount of weak acid sites and strength were reduced after SAPO-34 growth on both 3D-printed monoliths. This implies that the increase in total acid sites amount was from the increase in strong acid sites amount. In FIG. 22B, the IR peaks appeared at around 1540 and 1450 cm⁻¹ are attributed to the Brønsted (B) and Lewis (L) acid sites, respectively; whereas the band at around 1488 cm⁻¹ is regarded as the combination of B and L acid sites [49]. Corresponding Brønsted and Lewis acid sites amounts calculated from the py-FTIR intensity are tabalated in Table 4, which imply that formulation into monolith resulted in less Brønsted acid sites and slightly more Lewis acid sites. The reduction of the total acid sites by incorporation of silica is mainly on Brønsted acid. Both Brønsted and Lewis acid sites were increased after the SAPO-34 growth.

TABLE 4
Acid site distribution of the 3D-printed monoliths.

| | Weak | | Strong | | Total | L | B |
|---|---|---|---|---|---|---|---|
| | acid peak | | acid peak | | Acid | acid | acid |
| | | Amount | | Amount | amount | site | site |
| | T | (mmol | T | (mmol | (mmol | (mmol | (mmol |
| Sample | (K) | g⁻¹) | (K) | g⁻¹) | g⁻¹) | g⁻¹) | g⁻¹) |
| ZP | 490 | 0.268 | 676 | 0.309 | 0.577 | 0.174 | 0.403 |
| M1 | 487 | 0.365 | 651 | 0.205 | 0.570 | 0.194 | 0.376 |
| SPM1 | 481 | 0.352 | 654 | 0.277 | 0.629 | 0.220 | 0.409 |
| M2 | 479 | 0.220 | 666 | 0.018 | 0.239 | 0.119 | 0.120 |
| SPM2 | 477 | 0.202 | 633 | 0.161 | 0.363 | 0.151 | 0.212 |

Catalyst Testing:

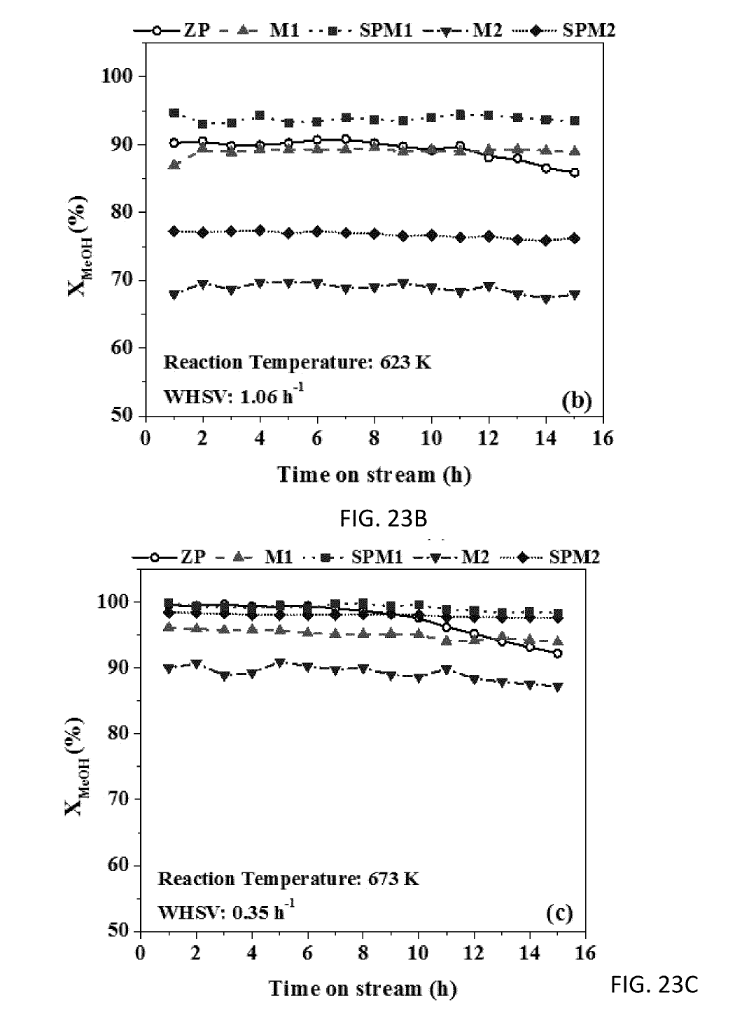

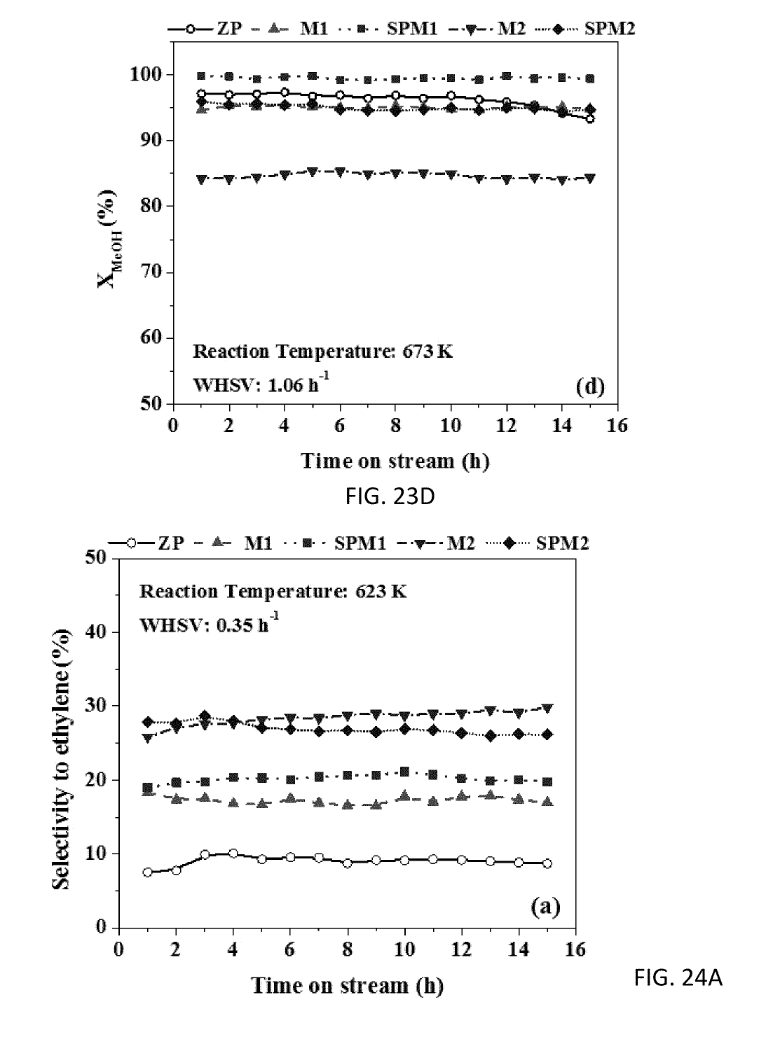

The catalytic performance of the 3D-printed monolithic catalysts and their parent HZSM-5 powder was tested in the MTO reaction at two temperatures and two contact times for 15 hours of time-on-stream and the methanol conversion (X_{MeOH}) results are shown in FIG. 23A, FIG. 23B, FIG. 23C, and FIG. 23D. All catalysts exhibited enhanced methanol conversion at higher temperature. HZSM-5 powder showed slightly higher conversion than its monolith counterpart M1 under all conditions within the initial stage of the reaction. This might be the consequence of the formation of mesopores and channels in monolith which promotes mass transfer through the catalyst bed. However, a notable decline in conversion was observed after around 12 hours time-on-stream, while M1 was found to be stable in the activity. The M2 monolith, on the other hand, showed much lower conversion under the same condition than M1 especially at 623 K due to its diluted concentration of zeolite. This is in accordance with the acidic property results obtained from NH₃-TPD measurements. Both M1 and M2 activities were enhanced by SAPO-34 crystals, according to the elevated methanol conversion rate of SPM1 and SPM2, resulted from the promising efficiency of SAPO-34 in methanol conversion [50]. All monoliths exhibited advantageous stability in catalytic MTO reaction compared to their HZSM-5 powder counterparts. Since it has been broadly accepted that the deactivation of zeolite catalysts in MTO conversion is mainly caused by coke formation occurred on confined zeolite framework cages and channels which block the active site [51, 52], it is proposed that the formulation of zeolite into the monolith structure reduces the coke deposition in MTO reaction as a result of hierarchical porous structure which favorers the mass transfer and enhances catalyst lifetime.

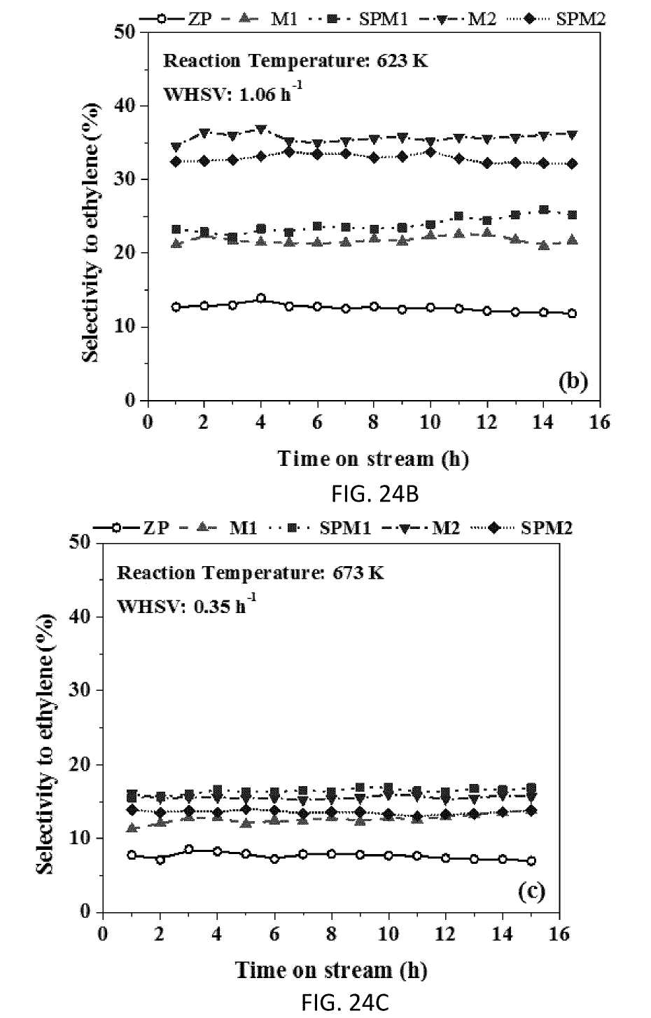

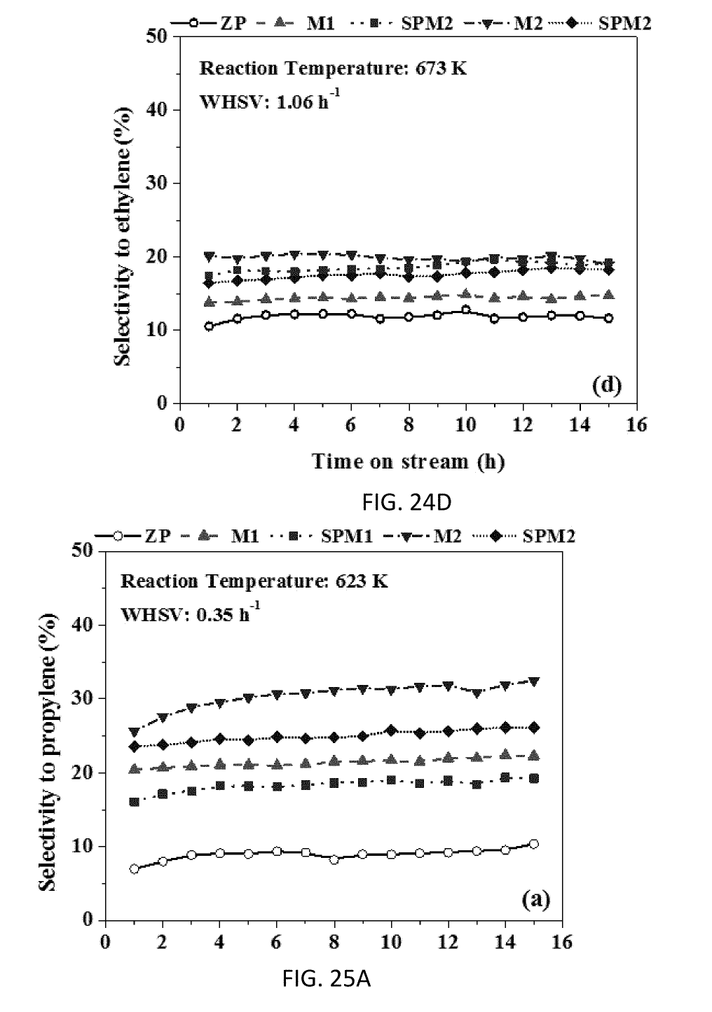

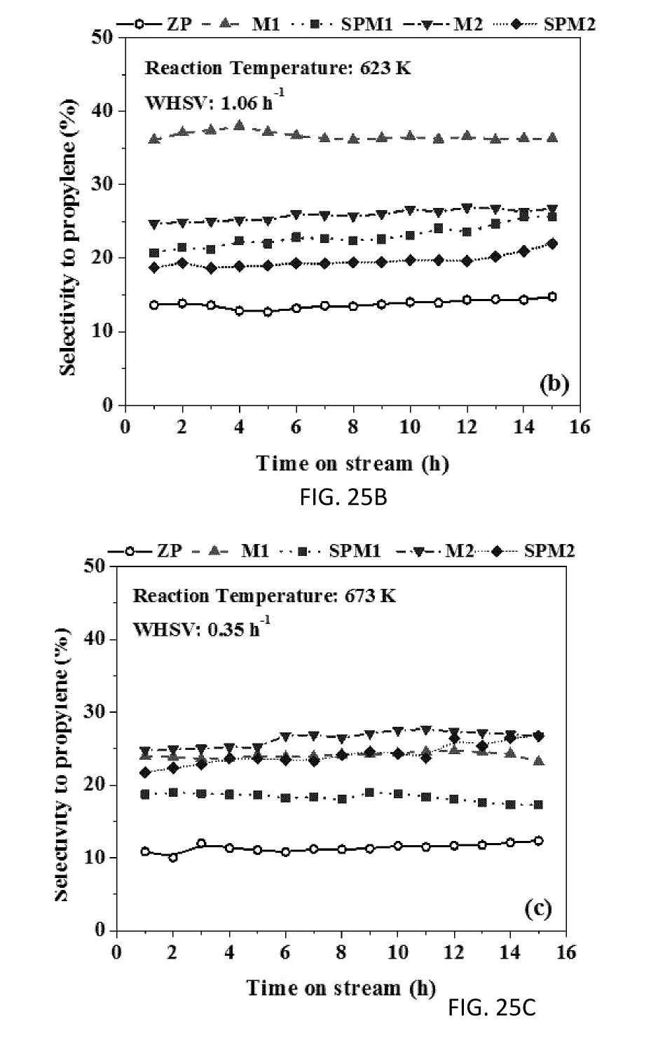

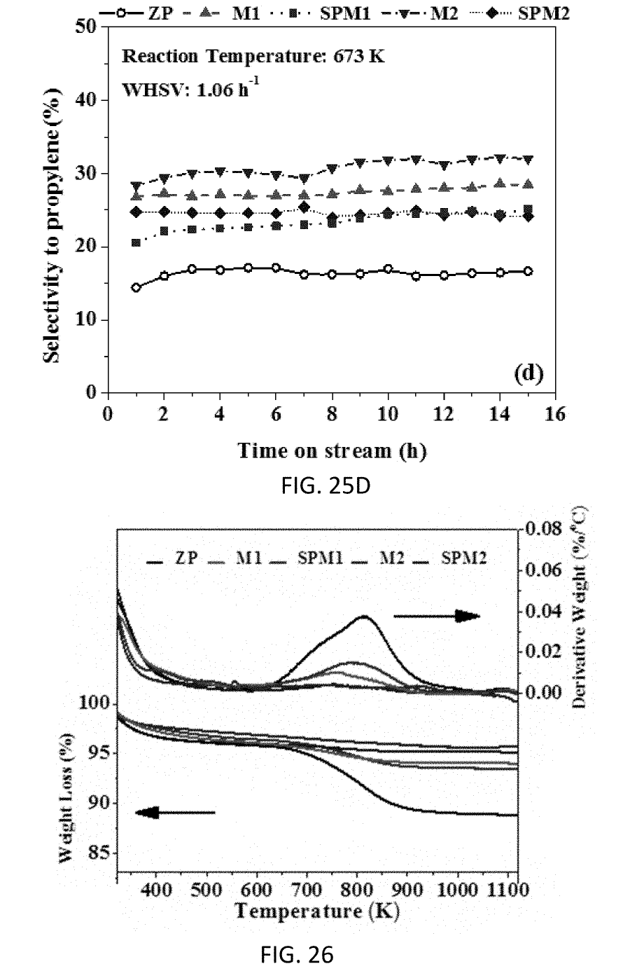

Moreover, the selectivity toward light olefins was enhanced over 3D-printed monoliths as shown in FIG. 24A, FIG. 24B, FIG. 24C, FIG. 24D, FIG. 25A, FIG. 25B, FIG. 25C, and FIG. 25D, in which the selectivity of ethylene and propylene were displayed as a function of time on stream respectively. Under all conditions, the 3D-printed monolith showed higher light olefin selectivity than the HZSM-5 powder. It is noted that the selectivity to ethylene over M2 at 623 K and under the space velocity of 1.02 h⁻¹ increased to ca. 37.1% whereas the HZSM-5 powder showed 13.6% selectivity at the same condition. It is widely accepted that factors such as reaction temperature, space velocity, catalyst acid property, and intermediate/product mass transfer can significantly affect the product distribution of a heterogeneous catalytic reaction. The remarkably high ethylene selectivity of M2 under this condition stems from the combination of mild reaction temperature [53], moderate acidity [54], hierarchical porosity [55], and promoted intermediate/product mass transfer [56]. Although, considering the methanol conversion rate of ca. 68.6% at this condition, the total throughput of ethylene is limited, the strategy of silica incorporation into the zeolite monolith is promising, given the benefits of more robust strengthened structure with less fabrication cost. The effect of SAPO-34 on selectivity to ethylene and propylene was found to be different. SAPO-34 tends to increase ethylene selectivity at all reaction conditions whereas the propylene selectivity remains at the same level, or even abated slightly at high temperature. This might be attributed to the fact that chance of ethylene, with a kinetic diameter of 4.0 Å, to transfer through SAPO-34 molecular sieve of 3.8 Å channel is are higher than propylene, with kinetic diameter of 4.0 Å [57].

Besides ethylene and propylene, the products generated from methanol conversion over the zeolite catalysts mainly consisted of butylene, paraffin (C₁-C₄), BTX (benzene, toluene, and xylene) and other hydrocarbons with C₅₊, as determined by the GC. The distribution of the products of 5 hours on stream over various catalysts under different conditions are listed in Table 5. It is important to emphasize here that BTX selectivity over HZSM-5 powder was significantly higher than the other byproducts. It has been previously proven that aromatic hydrocarbons such as BTX compounds are the precursors of coke formation in MTO over zeolites [58-60]. FIG. 26 displays the TGA and the corresponding DTA profiles for the investigated catalysts after 16 hours of MTO reaction at 673 K with a WHSV of 0.35 h⁻¹. As evident, ZP showed a broad and strong DTA peak in the range of 623 to 973 K, indicating the formation of different species of polyaromatic, which are mainly heavy compounds of coke precursors. For monolith catalysts, M1 exhibited a much smaller amount of coke formation of around 1.1%, compared to its power catalyst of 3.8%. The peak position was shifted to lower temperature indicating that the coke formed over monolith catalyst was lighter. The fact that coke formation in SPM1 is slightly severer than in M2 can be attributed to the coverage of SAPO-34 crystals with smaller framework channels which confined the transport of the aromatics. A spot of coke formation observed in both M2 and SPM2 was due to their diluted acid site density and the limited methanol conversion over them.

TABLE 5
Hydrocarbon distribution over monolith
catalyst and the parent HZMS-powder.

| | | | | Hydrocarbon selectivity (%) | | | | | | |
|---|---|---|---|---|---|---|---|---|---|---|
| | | | | | | | Total | | | |
| | WHSV | T | X_{MeOH} | | | | Light | | C₁-C₄ | |
| Sample | (h⁻¹) | (K) | (%) | C₂₌ | C₃₌ | C₄₌ | Olefins | BTX | paraffin | Others |
| ZP | 0.35 | 623 | 91 | 9.1 | 8.9 | 3.7 | 21.8 | 19.0 | 51.6 | 7.8 |
| | | 673 | 98 | 7.7 | 11.7 | 4.1 | 23.5 | 17.2 | 53.0 | 6.5 |
| | 1.06 | 623 | 89 | 12.7 | 14.1 | 11.0 | 37.7 | 17.4 | 40.9 | 4.9 |
| | | 673 | 97 | 12.8 | 17.0 | 10.8 | 40.6 | 14.9 | 41.0 | 4.2 |
| M1 | 0.35 | 623 | 90 | 17.8 | 21.7 | 4.7 | 44.2 | 13.0 | 39.0 | 4.2 |
| | | 673 | 95 | 12.8 | 24.4 | 6.2 | 43.4 | 15.7 | 38.0 | 3.0 |
| | 1.06 | 623 | 89 | 22.4 | 36.5 | 4.5 | 63.4 | 8.4 | 24.2 | 4.4 |
| | | 673 | 95 | 14.9 | 27.5 | 5.2 | 47.6 | 10.5 | 38.0 | 4.2 |
| SPM1 | 0.35 | 623 | 98 | 21.1 | 19.0 | 2.1 | 42.2 | 14.7 | 39.4 | 3.7 |
| | | 673 | 100 | 16.9 | 18.8 | 4.2 | 39.9 | 9.5 | 45.0 | 5.7 |
| | 1.06 | 623 | 94 | 23.9 | 23.1 | 3.6 | 50.6 | 11.6 | 35.0 | 3.2 |
| | | 673 | 99 | 19.5 | 24.4 | 4.7 | 48.6 | 9.6 | 38.2 | 3.8 |
| M2 | 0.35 | 623 | 74 | 28.8 | 31.3 | 4.6 | 64.7 | 6.9 | 24.2 | 4.0 |
| | | 673 | 89 | 16.0 | 27.5 | 3.1 | 46.6 | 7.0 | 42.8 | 4.3 |
| | 1.06 | 623 | 69 | 35.2 | 26.6 | 2.8 | 64.7 | 12.6 | 18.2 | 4.7 |
| | | 673 | 85 | 19.3 | 31.8 | 3.2 | 54.4 | 11.2 | 31.0 | 4.0 |
| SPM2 | 0.35 | 623 | 85 | 26.9 | 25.8 | 2.1 | 54.8 | 10.7 | 36.4 | 2.8 |
| | | 673 | 98 | 13.3 | 24.3 | 2.5 | 40.1 | 10.8 | 45.3 | 4.0 |
| | 1.06 | 623 | 77 | 33.8 | 19.7 | 2.7 | 56.2 | 12.4 | 28.2 | 4.1 |
| | | 673 | 95 | 17.9 | 24.6 | 2.7 | 45.2 | 11.2 | 41.0 | 3.2 |

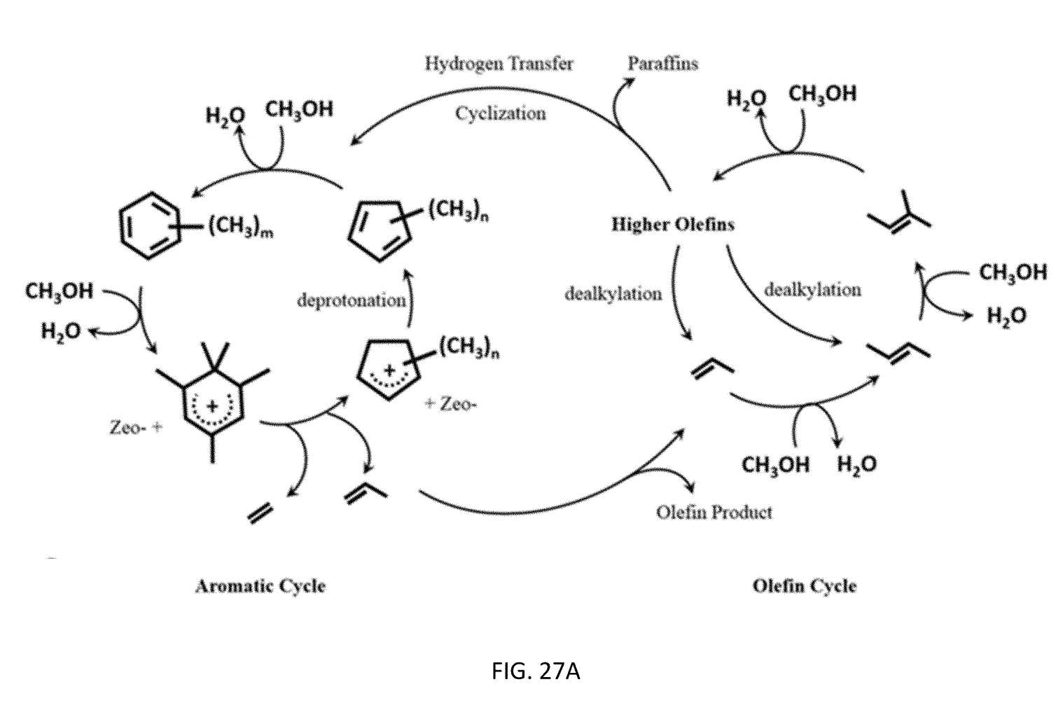

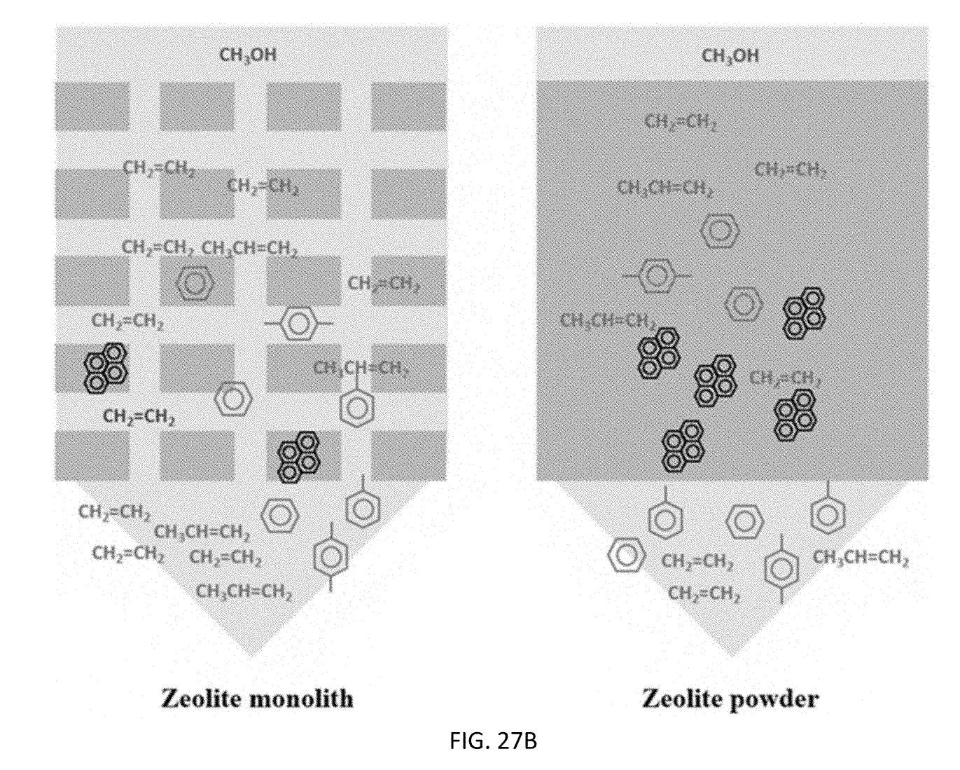

The hydrocarbon-pool mechanism of MTO process over zeolite, which has been proposed by Dahl et al. [61-63], is generally accepted, as shown in FIG. 27A. In this conversion over acidic catalyst, light olefins are formed from an equilibrium mixture of methanol and dimethyl ether. These olefins are methylated to higher olefins, which in turn are catalytically cracked again to lower olefins. Olefins, however, also react to paraffin and aromatics via hydrogen transfer and subsequently form coke [64]. Hydrogen transfer route on HZSM-5 takes place mostly between two olefin species on Brønsted acid sites, usually the strong acid [65]. M1 and M2 possess relatively low B/L (Brønsted to Lewis) ratio, hence mitigated the production of paraffin and aromatics. Although, the SAPO-34 enhanced the conversion by adding more acid cites, the additional Brønsted acid cites lowered the selectivity towards light olefins. Unlike to the powder HZSM-5 catalyst, the monoliths with hierarchal porosity offer rapid mass transfer through the mesopores and monolith channels, making the escape of the aromatics in the hydrocarbon pool easier, thus suppressing the growth of aromatics which later became the coke. This hypothesis has been verified by the TGA-DTA results of the spent catalysts and the analysis of products distribution. The schematic graph displayed in FIG. 27B illustrates the promoted MTO process over monolith catalyst in comparison with its powder counterpart.

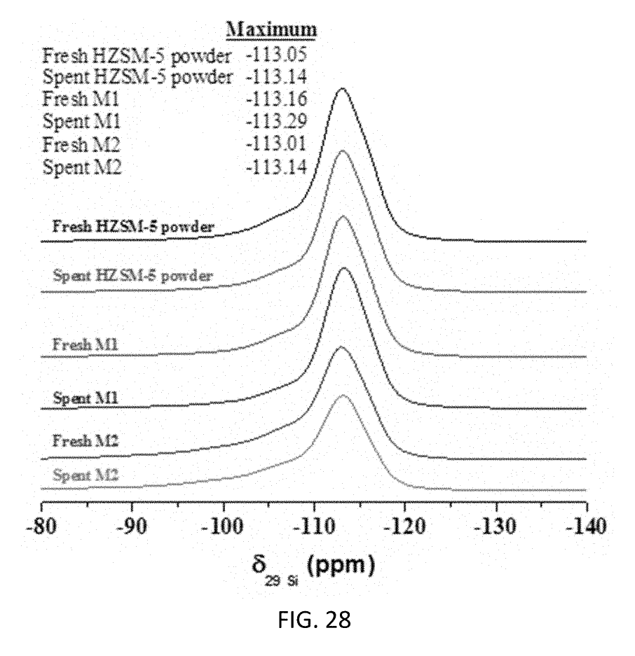

The ²⁹Si NMR spectra of the fresh and spent catalysts (HZMS-5 powder, M1, and M2) are shown in FIG. 28. The fresh HZSM-5 powder displayed a chemical shift at −113.05 ppm which was contributed from Q₄ of Si—(OSi)₄ to ca. −114.5 ppm and Q₃ of Si—(OSi)₃(OAl)₁ to ca. −105.8 ppm [66]. The displacement of the peak from −113.05 to −113.14 indicates the decrease in Si—(OSi)₃(OAl)₁ with respect to Si—(OSi)₄ [59]. A similar change was also observed on monolith catalysts with larger displacement. This change in chemical shift is resulted from the structural modification of the zeolite by dealumination and it is generally found in the steam-generating reactions such as methanol to hydrocarbons. A severer dealumination over monolith catalyst is the result of hierarchical porosity and promoted mass transfer. Interaction of steam with aluminum is more facile and thus caused structural modification. Taking the long reaction time of 15 h into consideration, the change in chemical shift of only 0.13 ppm is acceptable [59].

### Conclusion

This article described the experimental studies on the synthesis of customized 3D-printed zeolite monoliths with a hierarchical (macro-meso-microporous) pore network. The incorporation of amorphous silica and SAPO-34 crystal growth via secondary growth method were applied to tune the porosity and acidity of the zeolite monoliths. The incorporation of amorphous silica contributed to formation of additional mesopores and reduction in acid sites density. The growth of SAPO-34 caused pore clogging which reduced mesopores volume dramatically, whereas both Brønsted and Lewis acid sites were increased by the SAPO-34 crystals. Evaluation of the zeolite monoliths in MTO reaction indicated that the selectivity toward light olefins was favored by the novel 3D-printed structure as a result of modified acidity and porosity of the catalysts. Due to the reduced Brønsted acid site, the hydrogen transfer route in MTO reaction was mitigated and therefore production of paraffin and aromatic was suppressed and less coke formation was observed. Although slight dealumination was found on 3D-printed monoliths after MTO reaction, it was considered to be a stable structured catalyst due to its prolonged life time. This study provides a foundation for preparation of zeolite monoliths with tunable properties by 3D printing method that can be tailored for specific chemical reactions.

### REFERENCES

1. J. Cejka, A. Corma, S. Zones, Zeolites and catalysis: synthesis, reactions and applications, John Wiley & Sons, 2010.
2. M. Stöcker, Methanol-to-hydrocarbons: catalytic materials and their behavior, Microporous and Mesoporous Materials, 29 (1999) 3-48.
3. A. T. Aguayo, A. G. Gayubo, R. Vivanco, M. Olazar, J. Bilbao, Role of acidity and microporous structure in alternative catalysts for the transformation of methanol into olefins, Applied Catalysis A: General, 283 (2005) 197-207.
4. X. Li, A. Kant, Y. He, H. V. Thakkar, M. A. Atanga, F. Rezaei, D. K. Ludlow, A. A. Rownaghi, Light olefins from renewable resources: Selective catalytic dehydration of bioethanol to propylene over zeolite and transition metal oxide catalysts, Catalysis Today, 276 (2016) 62-77.
5. C. D. Chang, A. J. Silvestri, The conversion of methanol and other 0-compounds to hydrocarbons over zeolite catalysts, Journal of Catalysis, 47 (1977) 249-259.
6. B. P. C. Hereijgers, F. Bleken, M. H. Nilsen, S. Svelle, K.-P. Lillerud, M. Bjergen, B. M. Weckhuysen, U. Olsbye, Product shape selectivity dominates the Methanol-to-Olefins (MTO) reaction over H-SAPO-34 catalysts, Journal of Catalysis, 264 (2009) 77-87.
7. S. Svelle, F. Joensen, J. Nerlov, U. Olsbye, K.-P. Lillerud, S. Kolboe, M. Bjørgen, Conversion of Methanol into Hydrocarbons over Zeolite H-ZSM-5: Ethene Formation Is Mechanistically Separated from the Formation of Higher Alkenes, Journal of the American Chemical Society, 128 (2006) 14770-14771.
8. A. A. Rownaghi, F. Rezaei, J. Hedlund, Yield of gasoline-range hydrocarbons as a function of uniform ZSM-5 crystal size, Catalysis Communications, 14 (2011) 37-41.
9. J. C. Védrine, A. Auroux, P. Dejaifve, V. Ducarme, H. Hoser, S. Zhou, Catalytic and physical properties of phosphorus-modified ZSM-5 zeolite, Journal of Catalysis, 73 (1982) 147-160.
10. C. Mei, P. Wen, Z. Liu, H. Liu, Y. Wang, W. Yang, Z. Xie, W. Hua, Z. Gao, Selective production of propylene from methanol: Mesoporosity development in high silica HZSM-5, Journal of Catalysis, 258 (2008) 243-249.
11. A. A. Rownaghi, F. Rezaei, J. Hedlund, Uniform mesoporous ZSM-5 single crystals catalyst with high resistance to coke formation for methanol deoxygenation, Microporous and Mesoporous Materials 151 (2012) 26-33.
12. S. Ivanova, B. Louis, B. Madani, J. Tessonnier, M. Ledoux, C. Pham-Huu, ZSM-5 coatings on β-sic monoliths: possible new structured catalyst for the methanol-to-olefins process, The Journal of Physical Chemistry C, 111 (2007) 4368-4374.
13. A. A. Rownaghi, J. Hedlund, Methanol to Gasoline-Range Hydrocarbons: Influence of Nanocrystal Size and Mesoporosity on Catalytic Performance and Product Distribution of ZSM-5, Industrial & Engineering Chemistry Research, 50 (2011) 11872-11878.
14. A. A. Rownaghi, F. Rezaei, M. Stanteo, J. Hedlund, Selective dehydration of methanol to dimethyl ether on ZSM-5 nanocrystals, Applied Catalysis B: Environmental 119 (2011) 56-61.
15. T. Boger, A. K. Heibel, C. M. Sorensen, Monolithic catalysts for the chemical industry, Industrial & engineering chemistry research, 43 (2004) 4602-4611.
16. K. C. Taylor, Nitric oxide catalysis in automotive exhaust systems, Catalysis Reviews—Science and Engineering, 35 (1993) 457-481.
17. H. Gandhi, G. Graham, R. W. McCabe, Automotive exhaust catalysis, Journal of Catalysis, 216 (2003) 433-442.
18. F. Rezaei, A. A. Rownaghi, S. Monjezi, R. P. Lively, C. W. Jones, SOx/NOx removal from flue gas streams by solid adsorbents: a review of current challenges and future directions, Energy & fuels, 29 (2015) 5467-5486.
19. G. S. Bugosh, M. P. Harold, Impact of Zeolite Beta on Hydrocarbon Trapping and Light-Off Behavior on Pt/Pd/BEA/Al₂O₃ Monolith Catalysts, Emission Control Science and Technology, (2017) 1-12.
20. M. A. Buzanowski, R. T. Yang, Simple design of monolith reactor for selective catalytic reduction of NO for power plant emission control, Industrial and Engineering Chemistry Research; (United States), 29 (1990) 2074-2078.
21. P. S. Metkar, N. Salazar, R. Muncrief, V. Balakotaiah, M. P. Harold, Selective catalytic reduction of NO with NH3 on iron zeolite monolithic catalysts: Steady-state and transient kinetics, Applied Catalysis B: Environmental, 104 (2011) 110-126.
22. O. Kröcher, M. Devadas, M. Elsener, A. Wokaun, N. Sager, M. Pfeifer, Y. Demel, L. Mussmann, Investigation of the selective catalytic reduction of NO by NH3 on Fe-ZSM5 monolith catalysts, Applied Catalysis B: Environmental, 66 (2006) 208-216.
23. F. Notoya, C. Su, E. Sasaoka, S. Nojima, Effect of SO2 on the low-temperature selective catalytic reduction of nitric oxide with ammonia over TiO2, ZrO2, and Al2O3, Industrial & engineering chemistry research, 40 (2001) 3732-3739.
24. M. Behl, S. Roy, Experimental investigation of gas-liquid distribution in monolith reactors, Chemical Engineering Science, 62 (2007) 7463-7470.
25. B. Li, Z. Hu, B. Kong, J. Wang, W. Li, Z. Sun, X. Qian, Y. Yang, W. Shen, H. Xu, D. Zhao, Hierarchically tetramodal-porous zeolite ZSM-5 monoliths with template-free-derived intracrystalline mesopores, Chemical Science, 5 (2014) 1565-1573.
26. Y.-J. Lee, Y.-W. Kim, K.-W. Jun, N. Viswanadham, J. W. Bae, H.-S. Park, Textural Properties and Catalytic Applications of ZSM-5 Monolith Foam for Methanol Conversion, Catalysis Letters, 129 (2009) 408-415.
27. J. L. Williams, Monolith structures, materials, properties and uses, Catalysis Today, 69 (2001) 3-9.
28. J. Freiding, B. Kraushaar-Czarnetzki, Novel extruded fixed-bed MTO catalysts with high olefin selectivity and high resistance against coke deactivation, Applied Catalysis A: General, 391 (2011) 254-260.
29. M. Menges, B. Kraushaar-Czarnetzki, Kinetics of methanol to olefins over AlPO4-bound ZSM-5 extrudates in a two-stage unit with dimethyl ether pre-reactor, Microporous and Mesoporous Materials, 164 (2012) 172-181.
30. [30] A. Kant, Y. He, A. Jawad, X. Li, F. Rezaei, J. D. Smith, A. A. Rownaghi, Hydrogenolysis of glycerol over Ni, Cu, Zn, and Zr supported on H-beta, Chemical Engineering Journal, 317 (2017) 1-8.
31. C. R. Tubío, J. Azuaje, L. Escalante, A. Coelho, F. Guitián, E. Sotelo, A. Gil, 3D printing of a heterogeneous copper-based catalyst, Journal of Catalysis, 334 (2016) 110-115.
32. J. Lefevere, M. Gysen, S. Mullens, V. Meynen, J. Van Noyen, The benefit of design of support architectures for zeolite coated structured catalysts for methanol-to-olefin conversion, Catalysis Today, 216 (2013) 18-23.
33. H. Thakkar, S. Eastman, A. Hajari, A. A. Rownaghi, J. C. Knox, F. Rezaei, 3D-Printed Zeolite Monoliths for CO2 Removal from Enclosed Environments, ACS Applied Materials & Interfaces, 8 (2016) 27753-27761.
34. H. Thakkar, S. Eastman, A. Al-Mamoori, A. Hajari, A. A. Rownaghi, F. Rezaei, Formulation of Aminosilica Adsorbents into 3D-Printed Monoliths and Evaluation of Their CO2 Capture Performance, ACS Applied Materials & Interfaces, 9 (2017) 7489-7498.
35. H. Thakkar, S. Eastman, Q. Al-Naddaf, A. A. Rownaghi, F. Rezaei, 3D-Printed Metal-Organic Framework Monoliths for Gas Adsorption Processes, ACS Applied Materials & Interfaces, 9 (2017) 35908-35916.
36. J. Lefevere, L. Protasova, S. Mullens, V. Meynen, 3D-printing of hierarchical porous ZSM-5: The importance of the binder system, Materials & Design, 134 (2017) 331-341.
37. X. Li, W. Li, F. Rezaei, A. Rownaghi, Catalytic cracking of n-hexane for producing light olefins on 3D-printed monoliths of MFI and FAU zeolites, Chemical Engineering Journal, 333, (2018) 545-553.
38. K. Ma, H. Yu, G. Feng, C. Wang, Y. Dai, Synthesis of ZSM-5@ ordered mesoporous silica composites by dodecylamine surfactant, Journal of Wuhan University of Technology-Mater. Sci. Ed., 29 (2014) 1124-1128.
39. R. M. Jacobberger, B. Kiraly, M. Fortin-Deschenes, P. L. Levesque, K. M. McElhinny, G. J. Brady, R. Rojas Delgado, S. Singha Roy, A. Mannix, M. G. Lagally, P. G. Evans, P. Desjardins, R. Martel, M. C. Hersam, N. P. Guisinger, M. S. Arnold, Direct oriented growth of armchair graphene nanoribbons on germanium, Nature Communications, 6 (2015) 8006.
40. J. K. Das, N. Das, S. Bandyopadhyay, Highly oriented improved SAPO 34 membrane on low cost support for hydrogen gas separation, Journal of Materials Chemistry A, 1 (2013) 4966-4973.
41. M. A. Carreon, S. Li, J. L. Falconer, R. D. Noble, Alumina-Supported SAPO-34 Membranes for CO2/CH4 Separation, Journal of the American Chemical Society, 130 (2008) 5412-5413.
42. M. Li, J. Zhang, X. Liu, Y. Wang, C. Liu, D. Hu, G. Zeng, Y. Zhang, W. Wei, Y. Sun, Synthesis of high performance SAPO-34 zeolite membrane by a novel two-step hydrothermal synthesis+dry gel conversion method, Microporous and Mesoporous Materials, 225 (2016) 261-271.
43. X. Li, F. Rezaei, D. K. Ludlow, A. A. Rownaghi, Synthesis of SAPO-34@ZSM-5 and SAPO-34@Silicalite-1 Core-Shell Zeolite Composites for Ethanol Dehydration, Industrial & Engineering Chemistry Research, 57 (2018) 1446-1453.
44. M. Hartmann, W. Schwieger, Hierarchically-structured porous materials: from basic understanding to applications, Chemical Society Reviews, 45 (2016) 3311-3312.
45. M. Hartmann, A. G. Machoke, W. Schwieger, Catalytic test reactions for the evaluation of hierarchical zeolites, Chemical Society Reviews, 45 (2016) 3313-3330.
46. D. Liu, A. Bhan, M. Tsapatsis, S. Al Hashimi, Catalytic Behavior of Brønsted Acid Sites in MWW and MFI Zeolites with Dual Meso- and Microporosity, ACS Catalysis, 1 (2011) 7-17.
47. L. Emdadi, Y. Wu, G. Zhu, C.-C. Chang, W. Fan, T. Pham, R. F. Lobo, D. Liu, Dual Template Synthesis of Meso- and Microporous MFI Zeolite Nanosheet Assemblies with Tailored Activity in Catalytic Reactions, Chemistry of Materials, 26 (2014) 1345-1355.
48. C. A. R. Costa, L. F. Valadares, F. Galembeck, Stober silica particle size effect on the hardness and brittleness of silica monoliths, Colloids and Surfaces A: Physicochemical and Engineering Aspects, 302 (2007) 371-376.
49. V. S. Marakatti, A. B. Halgeri, Metal ion-exchanged zeolites as highly active solid acid catalysts for the green synthesis of glycerol carbonate from glycerol, RSC Advances, 5 (2015) 14286-14293.
50. L. Zhang, Y. Huang, New Insights into Formation of Molecular Sieve SAPO-34 for MTO Reactions, The Journal of Physical Chemistry C, 120 (2016) 25945-25957.
51. S. Müller, Y. Liu, M. Vishnuvarthan, X. Sun, A. C. van Veen, G. L. Haller, M. Sanchez-Sanchez, J. A. Lercher, Coke formation and deactivation pathways on H-ZSM-5 in the conversion of methanol to olefins, Journal of Catalysis, 325 (2015) 48-59.
52. P. Tian, Y. Wei, M. Ye, Z. Liu, Methanol to Olefins (MTO): From Fundamentals to Commercialization, ACS Catalysis, 5 (2015) 1922-1938.
53. T. Liang, J. Chen, Z. Qin, J. Li, P. Wang, S. Wang, G. Wang, M. Dong, W. Fan, J. Wang, Conversion of Methanol to Olefins over H-ZSM-5 Zeolite: Reaction Pathway Is Related to the Framework Aluminum Siting, ACS Catalysis, 6 (2016) 7311-7325.
54. A. Takahashi, W. Xia, I. Nakamura, H. Shimada, T. Fujitani, Effects of added phosphorus on conversion of ethanol to propylene over ZSM-5 catalysts, Applied Catalysis A: General, 423-424 (2012) 162-167.
55. Y. Gao, B. Zheng, G. Wu, F. Ma, C. Liu, Effect of the Si/AI ratio on the performance of hierarchical ZSM-5 zeolites for methanol aromatization, RSC Advances, 6 (2016) 83581-83588.
56. B. Liu, D. Slocombe, M. AlKinany, H. AlMegren, J. Wang, J. Arden, A. Vai, S. Gonzalez-Cortes, T. Xiao, V. Kuznetsov, P. P. Edwards, Advances in the study of coke formation over zeolite catalysts in the methanol-to-hydrocarbon process, Applied Petrochemical Research, 6 (2016) 209-215.
57. M. W. Anderson, B. Sulikowski, P. J. Barrie, J. Klinowski, In situ solid-state NMR studies of the catalytic conversion of methanol on the molecular sieve SAPO-34, The Journal of Physical Chemistry, 94 (1990) 2730-2734.
58. E. Epelde, J. I. Santos, P. Florian, A. T. Aguayo, A. G. Gayubo, J. Bilbao, P. Castaño, Controlling coke deactivation and cracking selectivity of MFI zeolite by H3PO4 or KOH modification, Applied Catalysis A: General, 505 (2015) 105-115.
59. M. Ibanez, M. Gamero, J. Ruiz-Martinez, B. M. Weckhuysen, A. T. Aguayo, J. Bilbao, P. Castano, Simultaneous coking and dealumination of zeolite H-ZSM-5 during the transformation of chloromethane into olefins, Catalysis Science & Technology, 6 (2016) 296-306.
60. V. Van Speybroeck, K. De Wispelaere, J. Van der Mynsbrugge, M. Vandichel, K. Hemelsoet, M. Waroquier, First principle chemical kinetics in zeolites: the methanol-to-olefin process as a case study, Chemical Society Reviews, 43 (2014) 7326-7357.
61. I. M. Dahl, S. Kolboe, On the reaction mechanism for propene formation in the MTO reaction over SAPO-34, Catal Lett, 20 (1993) 329-336.
62. I. M. Dahl, S. Kolboe, On the reaction mechanism for hydrocarbon formation from methanol over SAPO-34: I: isotopic labeling studies of the co-reaction of ethene and methanol, J Catal, 149 (1994) 458-464.
63. I. M. Dahl, S. Kolboe, On the Reaction Mechanism for Hydrocarbon Formation from Methanol over SAPO-34: 2. Isotopic Labeling Studies of the Co-reaction of Propene and Methanol, Journal of Catalysis, 161 (1996) 304-309.
64. S. Müller, Y. Liu, F. M. Kirchberger, M. Tonigold, M. Sanchez-Sanchez, J. A. Lercher, Hydrogen Transfer Pathways during Zeolite Catalyzed Methanol Conversion to Hydrocarbons, Journal of the American Chemical Society, 138 (2016) 15994-16003.
65. S. Ilias, A. Bhan, Mechanism of the Catalytic Conversion of Methanol to Hydrocarbons, ACS Catalysis, 3 (2013) 18-31.
66. S. A. Axon, J. Klinowski, Solid-State NMR Studies of Zeolite [Si,B]-ZSM-5 Synthesized by the “Fluoride Method”, The Journal of Physical Chemistry, 98 (1994) 1929-1932.

All cited references are herein expressly incorporated by reference in their entirety.

Whereas particular embodiments have been described above for purposes of illustration, it will be appreciated by those skilled in the art that numerous variations of the details may be made without departing from the disclosure as described in the appended claims.

What is claimed is:

1. A zeolite coated monolith article comprising an uncoated monolithic support structure including walls having a honeycomb structure comprising ammonia-ZSM-5 powder (SiO₂/Al₂O₃) and bentonite clay; and a porous coating disposed directly upon the uncoated monolithic support structure.

2. The zeolite coated monolith article of claim 1, further comprises an additional component selected from the group consisting of amorphous silica, a plasticizing binder, a metal dopant, and combinations thereof disposed within the uncoated monolithic support structure.

3. The zeolite coated monolith article of claim 2, wherein the metal dopant is selected from the group consisting of Zn, Ce, Cr, Mg, Cu, La, Ga, Y, and combinations thereof.

4. The zeolite coated monolith article of claim 1, wherein the porous coating comprises SAPO-34.

5. The zeolite coated monolith article of claim 1, wherein the zeolite coated monolith has a wall thickness of about 0.2 mm to about 0.9 mm.

6. The zeolite coated monolith article of claim 1, wherein the zeolite coated monolith has a square channel length of about 0.2 mm to about 1.6 mm.

7. The zeolite coated monolith article of claim 1, wherein the zeolite coated monolith has a total pore volume of about 0.2 cm³/g to about 0.95 cm³/g.

8. The zeolite coated monolith article of claim 1, wherein the zeolite coated monolith has a mesoporosity of about 0.1 cm³/g to about 0.95 cm³/g.

9. A process for converting methanol to one or more light olefins (MTO), the process comprising contacting methanol, under deoxygenation conditions, with a catalyst comprising a zeolite monolith,
wherein the zeolite monolith comprises an uncoated monolithic support structure including walls having a honeycomb structure comprising ammonia-ZSM-5 powder (SiO₂/Al₂O₃) and bentonite clay, and a porous coating disposed directly upon the uncoated monolithic support structure.

10. The process of claim 9, wherein the one or more light olefins is selected from the group consisting of ethylene, propylene, butylene, and combinations thereof.

11. The process of claim 9, wherein the process occurs in a tubular reactor.

12. The process of claim 11, wherein the tubular reactor a fixed bed reactor or a fluidized-bed reactor.

13. The process of claim 12 wherein the tubular reactor is a fixed bed reactor.

14. A process for catalytic cracking a hydrocarbon to produce a light olefin, the process comprises contacting the hydrocarbon, under cracking conditions, with a catalyst comprising a zeolite monolith,
wherein the zeolite monolith comprises an uncoated monolithic support structure including walls having a honeycomb structure comprising ammonia-ZSM-5 powder (SiO₂/Al₂O₃) and bentonite clay, and a porous coating disposed directly upon the uncoated monolithic support structure.

15. The process of claim 14, wherein the process occurs at a temperature from about 550° C. to about 700° C.

16. The process of claim 14, wherein the process occurs at pressure from about 0.5 bar to about 2 bar.

17. The process of claim 14, wherein the hydrocarbon is selected from the group consisting of ethane, propane, butane, pentane, and hexane.

18. The process of claim 17, wherein the hydrocarbon is n-hexane.

19. The process of claim 14, wherein the light olefin is selected from the group consisting of ethylene, propylene, butylene, and combinations thereof.

20. The process of claim 14, wherein the process occurs in a tubular reactor and the tubular reactor is a fixed bed reactor or a fluidized-bed reactor.
